(19) United States
(12) Patent Application Publication
(10) Pub. No.: US 2010/0063268 A1
(43) Pub. Date: Mar. 11, 2010

(54) NUCLEIC ACID EXTRACTION METHOD

(76) Inventors: Hideyuki Kanehara, Saitama (JP); Tasuku Sasaki, Saitama (JP)

Correspondence Address: BIRCH STEWART KOLASCH & BIRCH, PO BOX 747, FALLS CHURCH, VA, 22040-0747, US

(21) Appl. No.: 11/993,352

(22) PCT Filed: Sep. 28, 2006
(86) PCT No.: PCT/JP2006/319903

§ 371 Date: Dec. 20, 2007

(30) Foreign Application Priority Data
Sep. 28, 2005 (JP) 2005-282130
Sep. 27, 2006 (JP) 2005-263384

(51) Int. Cl. C07H 21/00

(52) U.S. Cl. 536/55.3

(57) ABSTRACT

A method for extracting a nucleic acid, which comprises: (a) preparing a biomaterial containing a solution by a following step (i) or (ii): (i) a step in which a biomaterial containing a phosphate buffer solution or a Bis-Tris (N,N-bis(2-hydroxyethyl)iminotris(hydroxymethyl)methane) buffer solution is prepared; or (ii) a step in which a buffer solution contained in a biomaterial is replaced with a Bis-Tris buffer solution; (b) dissolving the biomaterial with a lysis solution, and eluting a nucleic acid contained in the biomaterial; (c) preparing a lysate solution by adding a water-soluble organic solvent to the nucleic acid-eluted solution obtained in the step (b); (d) allowing the nucleic acid contained in the lysate solution to be adsorbed by a solid material; (e) washing impurities remaining in the solid material and the lysis solution; and (f) desorbing the absorbed nucleic acid from the solid material by a recovering solution.

### TECHNICAL FIELD

This invention relates to a method for extracting a nucleic acid from a biomaterial.

### BACKGROUND ART

The nucleic acid extraction method is mainly divided into two types, namely a method in which the extraction is carried out in a state of solution and a solid material-mediated method in which a nucleic acid is absorbed by allowing a solution containing the nucleic acid to contact with the solid material, washed and then desorbed.

Among the methods which are carried out in a state of solution, the extraction method which has been carried out from the most old times is a method which is carried out by clinging a nucleic acid precipitated with ethanol to a glass rod. This method is very convenient but has a big problem in terms of the yield and purity.

As the method for improving these problems, in the case of the extraction of RNA for example, an AGPC (acid guanidinium phenol chloroform) method described in the P. D. Siebert and A. Chenchik, Nucleic Acids Res., 21, 2019-2020 (1993) in which cells are lysed by adding guanidine thiocyanate and then coexisting DNA is removed using phenol under an acidic condition, and a guanidine-cesium chloride centrifugation method which uses high floating density of RNA in comparison with DNA and protein are known. However, these methods have many disadvantages such as the use of toxic organic compounds such as phenol and chloroform, the difficulty in recovering the RNA having small molecular weight, the requirement of complex operations, and the requirement of skilled techniques for carrying out precise extraction.

As a method for improving these disadvantages, a method has been developed in which a lysate solution prepared by lysing cells and adding ethanol thereto using guanidine thiocyanate or the like chaotropic salt which inhibits a nuclease or the like enzyme capable of accelerating degradation of a nucleic acid is allowed to contact with silica or the like solid material that absorbs the nucleic acid, the material is washed and then the nucleic acid is desorbed (R. Boom et al., Journal of Clinical Microbiology, 28, 495-503 (1990)).

As another method, a nucleic acid extraction method which uses magnetic silica particles has been developed and improvement of its reaction efficiency and washing efficiency has been carried out. In addition, a nucleic acid extraction method which uses a porous membrane has been developed (JP-A-2003-128691), so that it became possible to obtain a high purity nucleic acid by a convenient method within a short period of time.

### DISCLOSURE OF THE INVENTION

Accordingly, an object of the invention is to provide a method for separating and purifying a nucleic acid in which a nucleic acid in an analyte is allowed to be absorbed by a solid phase surface and then desorbed via washing and the like steps. Another object of the invention is to provide a method for separating and purifying a nucleic acid using a solid phase, which has excellent separation performance and good washing efficiency, can be easily processed, and can mass-produce those which have substantially the same separation performance, and a nucleic acid separation purification unit which is suited for carrying out the method. A still another object of the invention is to separate and purify a nucleic acid conveniently and quickly using a small device which does not require a special technique, a complex operation and a special device. A further object of the invention is to improve pass-through rate of a lysate solution and washing solution while keeping high yield and high purity, effected by improving clogging at the time of extraction operation.

The invention aims at separating and purifying a nucleic acid by lysing a biomaterial, and allowing the nucleic acid component contained in the biomaterial to contact with a container prepared by immobilizing a solid material such as a porous membrane into a container such as a cartridge. It relates to a method to be used in such a case for shortening pass-through time of a lysate solution and washing solution, and at the same time for effecting pass-through of cell species and the number of cells without causing clogging, which were unable to treat by the conventional methods due to clogging. In order to develop such a method, examination was made using a dispersion medium, particularly a phosphate buffer or Bis-Tris buffer, on various conditions such as the concentration of a surface active agent and a chaotropic salt in a lysis solution having the action to lyse biomaterials, pipetting after the addition of the lysis solution, stirring after the addition of a water-soluble organic solvent, a cartridge in which a solid material is sealed, for example, a cartridge for separation and purification of nucleic acid prepared by sealing a porous membrane in a container having two openings, and pore size of the membrane sealed in this cartridge, and it was found that the aforementioned objects can be attained by jointly using these conditions, thus resulting in the accomplishment of the invention. That is, the invention consists of the following constructions.

(1) A method for extracting a nucleic acid, which comprises:

(a) preparing a biomaterial containing a solution by a following step (i) or (ii):
(i) a step in which a biomaterial containing a phosphate buffer solution or a Bis-Tris (N,N-bis(2-hydroxyethyl)iminotris(hydroxymethyl)methane) buffer solution is prepared; or
(ii) a step in which a buffer solution contained in a biomaterial is replaced with a Bis-Tris buffer solution;

(b) dissolving the biomaterial by allowing the biomaterial to contact with a lysis solution, and eluting a nucleic acid contained in the biomaterial;

(c) preparing a lysate solution by adding a water-soluble organic solvent to the nucleic acid-eluted solution obtained in the step (b);

(d) allowing the nucleic acid contained in the lysate solution to be adsorbed by a solid material by allowing the lysate solution to contact with the solid material;

(e) washing impurities remaining in the solid material, other than the nucleic acid to be extracted, and the lysis solution; and

(f) desorbing the absorbed nucleic acid from the solid material by a recovering solution.

(2) The nucleic acid extraction method as described in (1) above,

wherein the solution in the step (a) is a dispersion medium.

(3) The nucleic acid extraction method as described in (1) or (2) above,

wherein the solution in the step (a) has a concentration of from 0.01 to 10 mol/l and pH of from 3 to 9.

(4) The nucleic acid extraction method as described in any of (1) to (3) above,

wherein the lysis solution in the step (b) contains a chaotropic salt.

(5) The nucleic acid extraction method as described in (4) above,

wherein a concentration of the chaotropic salt is from 0.1 to 10 mol/l.

(6) The nucleic acid extraction method as described in any of (1) to (5) above,

wherein the lysis solution in the step (b) contains a water-soluble organic solvent in a concentration of 50% by volume or less.

(7) The nucleic acid extraction method as described in (6) above,

wherein the water-soluble organic solvent contained in the lysis solution is methanol, ethanol, isopropanol or butanol.

(8) The nucleic acid extraction method as described in any of (1) to (7) above,

wherein the lysis solution in the step (b) contains a surface active agent.

(9) The nucleic acid extraction method as described in (8) above,

wherein a concentration of the surface active agent contained in the lysis solution is from 0.001 to 30% by mass.

(10) The nucleic acid extraction method as described in any of (1) to (9) above,

wherein at least one pipetting operation is carried out after adding the lysis solution in the step (b).

(11) The nucleic acid extraction method as described in any of (1) to (10) above,

wherein stirring is carried out after the addition of the lysis solution or after the pipetting operation in the step (b).

(12) The nucleic acid extraction method as described in any of (1) to (11) above,

wherein the lysate solution in the step (c) is prepared by adding a water-soluble organic solvent to the nucleic acid-containing lysis solution so that the lysate solution contains the water-soluble organic solvent in a concentration of from 10% by volume to 60% by volume.

(13) The nucleic acid extraction method as described in (12) above,

wherein the water-soluble organic solvent to be used in the step (c) is methanol, ethanol, isopropanol or butanol.

(14) The nucleic acid extraction method as described in any of (1) to (13) above,

wherein at least one pipetting operation or stirring is carried out in the step (c) after the addition of the water-soluble organic solvent.

(15) The nucleic acid extraction method as described in (14) above,

wherein a stirring time is from 0.1 to 600 seconds.

(16) The nucleic acid extraction method as described in (14) or (15) above,

wherein after adding the water-soluble organic solvent and carrying out stirring or pipetting in the step (c), pipetting or stirring is further carried out.

(17) The nucleic acid extraction method as described in (16) above,

wherein a stirring time is from 0.1 to 600 seconds.

(18) The nucleic acid extraction method as described in any of (1) to (17) above,

wherein a soaking time of the recovering solution in the step (f) is from 0.1 second to 1,600 seconds.

(19) The nucleic acid extraction method as described in any of (1) to (18) above,

wherein when the number of cells is 500,000 or less, a liquid amount of the lysate solution to be used is 800 μl or less.

(20) The nucleic acid extraction method as described in any of (1) to (19) above,

wherein when the number of cells is 500,000 or more, a liquid amount of the lysate solution to be used is 300 μl or more.

(21) The nucleic acid extraction method as described in any of (1) to (20) above,

wherein the solid material in the step (d) is a solid material that has a hydroxyl group on a surface of the solid material.

(22) The nucleic acid extraction method as described in any of (1) to (21) above,

wherein a container in which the solid material is kept in a cartridge is used in the step (d).

(23) The nucleic acid extraction method as described in any of (1) to (22) above,

wherein the lysate solution is allowed to contact with the solid material through which a solution containing a chaotropic salt is passed in advance in the step (d).

(24) The nucleic acid extraction method as described in any of (1) to (23) above,

wherein an extraction is carried out by injecting the lysate solution into two or more of containers in the step (c).

(25) The nucleic acid extraction method as described in any of (1) to (24),

wherein the lysate solution is put twice or more into one cartridge in the step (c).

(26) The nucleic acid extraction method as described in any of (1) to (25) above,

wherein in the step (d), the step (e) and the step (f), at least one of the lysate solution, the washing solution and the recovering solution is allowed to contact with the solid material by a change of pressure or centrifugation.

(27) The nucleic acid extraction method as described in any of (1) to (26) above,

wherein the nucleic acid is one of DNA, RNA, mRNA and a plasmid or a mixture thereof.

(28) The nucleic acid extraction method as described in any of (1) to (27) above,

wherein the biomaterial is a cultured cell, an animal cell, an animal tissue, a plant cell, a plant tissue, a virus, a bacterium, a fungus or a nucleic acid.

### BRIEF DESCRIPTION OF THE DRAWING

FIG. 1 is a graph showing pass-through time when PBS buffer solution of PBS-containing pelletized HL 60 cells was replaced by 0.5 mol/l Bis-Tris buffer solution;

FIG. 2 is a graph showing relationship of the kind and volume of dispersion medium with the RNA recovery yield and pass-through time. The numerical value shown on the side of each buffer solution name is liquid volume of the buffer solution used in the dispersion (unit, μl). PBS is a solution containing 137 mmol/l of sodium chloride, 2.7 mmol/l of potassium chloride, 10 mmol/l of disodium hydrogenphosphate and 2 mmol/l of potassium dihydrogenphosphate;

FIG. 3 is a graph showing plotting of pass-through time of lysate and washing solution 1 and RNA recovery yield against concentration of 0.5 mol/l Bis-Tris buffer solution (pH 6.5);

FIG. 4 is a graph showing relationship of the kind of lysis solution with the pass-through time of lysate and washing solution 1 and RNA recovery yield;

FIG. 5 is a graph showing a relationship between the guanidine thiocyanate concentration and the RNA recovery yield;

FIG. 6 is a graph showing a relationship between the guanidine thiocyanate concentration and the pass-through time of washing solution;

FIG. 7 is a graph showing a relationship between the lysate solution volume and the pass-through time of lysate and washing solution 1 and RNA recovery yield;

FIG. 8 is a graph showing a relationship between the amount of ethanol in lysis solution and the pass-through time of lysate, washing solution 1 and washing solution 2;

FIG. 9 is a graph showing a relationship between the amount of ethanol in lysis solution and the RNA recovery yield by each recovering solution volume. The numerical value of from 100 μl to 700 μl shown on the right side is a total liquid volume when the recovering solution was added in 100 μl portions to one extraction column;

FIG. 10 is a graph showing a relationship between the concentration of ethanol in lysate and the RNA recovery yield;

FIG. 11 is a graph showing relationship of the stirring time after ethanol addition with the pass-through time of lysate and washing solution 1 and washing solution 2;

FIG. 12 is a graph showing a relationship between the stirring time after ethanol addition and the RNA recovery yield. The numerical value of from 100 μl to 300 μl shown on the right side is a total liquid volume when the recovering solution was added in 100 μl portions to one extraction column;

FIG. 13 is a graph showing relationship of the pore size of the membrane with the pass-through time of lysate and washing solution 1 and washing solution 2;

FIG. 14 is a graph showing a relationship between the pore size of the membrane and the RNA recovery yield;

FIG. 15 is a graph showing a relationship between the volume of lysate solution and the recovery yield; and

FIG. 16 is a graph showing nucleic acid recovery yield dependency when a solid phase is coated with lysis solution in advance.

### BEST MODE FOR CARRYING OUT THE INVENTION

When a biomaterial, for example, a nucleic acid component, is separated and purified from cells, a lysate solution is prepared by lysing the cells with a lysis solution containing a chaotropic salt and the like and adding a water-soluble organic solvent thereto, and when the nucleic acid dissolved in the lysis solution is aggregated, components derived from other than the nucleic acid are also aggregated and the lysate solution containing such aggregates are passed through a solid material such as a porous membrane in carrying out this, these components stop up pores in the porous membrane or are deposited on the pore surface so that pass-through time of the lysate solution and washing solution is prolonged. As a result, a possibility of generating clogging becomes high when the clogging components are frequent, for example, when the number of cells to be treated is large. According to the invention, these problems were solved by variously examining the lysate solution preparing method, extraction method, washing method and nucleic acid recovering method.

The nucleic acid extraction method of the invention comprises at least the following steps (a) to (f);

(a) a step in which a biomaterial is dispersed using a dispersion medium such as a buffer liquid (to be referred also to as “dispersion step” hereinafter),
(b) a step in which the biomaterial is dissolved by allowing the biomaterial to contact with a lysis solution, and the nucleic acid contained in the biomaterial is eluted (to be referred also to as “dissolution step” hereinafter),
(c) a step in which a lysate solution is prepared by adding a water-soluble organic solvent to the nucleic acid-eluted solution (to be referred also to as “lysate step” hereinafter),
(d) a step in which the nucleic acid contained in the lysis solution is allowed to be adsorbed by a solid material by allowing the lysate solution to contact with the solid material (to be referred also to as “adsorption step” hereinafter),
(e) a step in which the solid material is washed under the nucleic acid-adsorbed state using a wash liquid (to be referred also to as “washing step” hereinafter), and
(f) a step in which the nucleic acid is desorbed from the solid material by a recovering solution and discharged into outside moiety of the aforementioned cartridge container (to be referred also to as “recovery step” hereinafter).

Regarding the term “clogging” as used herein, a case in which the pass-through rate of the lysate solution or washing solution became 0, or a case in which the pass-through rate of the lysate solution or washing solution became a certain threshold value or more, for example, when it became 120 seconds or more, is called clogging.

When a nucleic acid is extracted from a biomaterial, it is desirable to disperse such a biomaterial in advance in an appropriate dispersion medium. In this case, when pelletized cells are used, they are frozen in many cases, so that it is desirable to thaw them from the viewpoint of improving dispersibility.

Regarding the kind of the dispersion medium, any substance can be used with the proviso that disruption and shrinkage of the biomaterial due to a difference in osmotic pressure are minimum, and it can disperse the cells. As such a solution, a buffer solution can for example be cited.

As the buffer agents, generally used pH buffer agents (buffers) can be exemplified. Particularly, pH buffer agents for biochemical use are desirable. As a result of intensive studies, the present inventors have revealed that, among buffer solutions, Bis-Tris (N,N-bis(2-hydroxyethyl)iminotris(hydroxymethyl)methane) buffer solution has short pass-through time and low possibility of causing clogging so that this can be suitably used.

Though amount of the dispersion medium is not particularly limited, it is desirable to use the dispersion medium by increasing its amount as the number of cells increases. However, when this is restricted by the amount of the lysate solution, the concentration or amount of the chaotropic salt having the ability to lyse a component of a biomaterial in the lysate, such as a cell, and also having the action to inhibit a nuclease such as RNase becomes small, thus increasing a possibility that the biomaterial cannot be dissolved sufficiently and a possibility that the yield is reduced because the action to inhibit activity of a nuclease having the action to degrade RNA or the like nucleic acid does not functions sufficiently. Accordingly, it is desirable that amount of the dispersion medium is as small as possible. On the other hand, when it is not restricted by the amount of lysate solution, it is possible to increase amount of the dispersion medium, but when amount of the dispersion medium is increased, it is necessary to increase amount of the lysate solution for keeping concentration of the chaotropic salt at a certain level or more, and increase of the amount of the lysate solution results in the increase of pass-through time, particularly in the prolongation of the pass-through time of the lysate solution, so that it is not desirable to unnecessarily increase amount of the lysate solution. Thus, it is desirable that the amount of the dispersion medium to be used in the invention is preferably 80% by volume or less, more preferably 50% by volume or less, most preferably 20% by volume or less, based on the amount of the lysate solution.

It is desirable to use the dispersion medium at such a concentration that the cells are not completely or partially degraded by the action of osmotic pressure or the like. Concentration of the dispersion medium exerts influence upon the pass-through time of the lysate solution and washing solution. For example, when Bis-Tris buffer (pH 6.5) is used as the dispersion medium and 30 μl is employed as the liquid volume, the pass-through time of the lysate solution and washing solution is prolonged when concentration of the dispersion medium is low. When concentration of the dispersion medium is high, there is a possibility that the cells are partially lysed, nucleic acids, proteins and the like inside the cells are eluted, and the nucleic acids are degraded by the action of the eluted nuclease and the like, thus resulting in the reduction of the yield of the nucleic acid of interest. Based on these, according to the invention, concentration of the dispersion medium is preferably 0.01 mol/l or more and 10 mol/l or less, more preferably 0.1 mol/l or more and 1 mol/l or less. The pH of the dispersion medium is preferably from 3 to 9, and more preferably from 5.5 to 8.5.

Amount (liquid volume) of the dispersion medium can be regulated based on the number of cells and cell species. For example, in the case of HL 60 and when the number of cells is 5,000,000 or less, the extraction can be carried out without using the dispersion medium from the viewpoint of the user's load alleviation, but when the dispersion medium is used, the reproducibility is good and the nucleic acid can be obtained with a high yield in many cases, so that it is desirable to use it. When the number of cells is 1,000,000 or more, it is desirable to use the dispersion medium. When the dispersion medium is not used or used only in a small amount, yield of the nucleic acid becomes low, and a possibility of generating dispersion of the yield becomes high in some cases. It is considered that the cause of this is because the cells formed a structure consisting of un-lysed materials in which the cells were not sufficiently lysed caused by the lysis solution added because of the high cell density, and as a result, a nucleic acid or the like component to be extracted was incorporated into the structure. Based on the above, according to the invention, it is desirable to use the dispersion medium, and it is particularly desirable to use the dispersion medium when the number of cells is large.

When pelletized cells are prepared, pass-through time of the lysate solution and washing solution at the time of the extraction can be sharply shortened by removing generally used PBS (phosphate buffered saline) as many as possible and changing it to Bis-Tris buffer, or by adding Bis-Tris buffer to the PBS-containing pelletized cells. Based on these, according to the invention, the cells can be re-dispersed by removing PBS or the like dispersion medium or component which causes prolongation of the pass-through time, and then adding other dispersion medium thereto.

The chaotropic salt in the lysis solution is not particularly limited, and any conventionally known chaotropic salt can be used. As the chaotropic salt, a guanidine salt, sodium isothiocyanate, sodium iodide, potassium iodide, urea, sodium bromide, potassium bromide, calcium bromide, ammonium isothiocyanate, sodium chloride, potassium chloride, ammonium chloride and the like can be used. Among them, a guanidine salt is desirable. As the guanidine salt, guanidine hydrochloride, guanidine isothiocyanate and guanidine thiocyanic acid salt (guanidine thiocyanate) can be exemplified, of which guanidine hydrochloride or guanidine thiocyanate is desirable. These salts may be used alone or as a combination of two or more.

As a result of intensive studies carried out by the inventors, concentration of the chaotropic salt is not particularly limited, with the proviso that a concentration which can effect sufficient lysis of the cells and short pass-through time of the prepared lysate solution and washing solution. The lysate solution prepared from a low concentration chaotropic salt was not able to lyse the cells, and though the pass-through time of lysate solution and washing solution was markedly quickened, it was completely unable to recover the nucleic acid. Regarding the reason for the quickened pass-through time, it was considered that the partially lysed cells did not enter into pores of the solid material but accumulated on the upper side of the solid material. In addition, this also increases a possibility that an enzyme capable of degrading the nucleic acid is eluted from the partially lysed biomaterial and degrades the nucleic acid to cause reduction of the yield. Based on these, it is desirable that concentration of the chaotropic salt to be used in the lysis solution is high.

When concentration of the chaotropic salt was increased, the biomaterial was gradually degraded, and recovery yield of the nucleic acid was increased accompanied by this, but pass-through time of the lysate solution and washing solution was also sharply prolonged. When the concentration was further increased, the cells were completely lysed at least by microscopic observation, pass-through time of the lysate solution and washing solution was shortened, and recovery yield of the nucleic acid was also improved sharply.

When concentration of the chaotropic salt was further increased, pass-through time of the lysate solution and washing solution was not sharply shortened, and recovery yield of the nucleic acid was almost constant. However, solubility of the lysis solution containing the chaotropic salt in water was reduced, so that preparation of the lysis solution became difficult, and a problem of causing precipitation of the chaotropic salt from the lysis solution prepared at a low temperature was generated.

Based on the above, it is desirable that concentration of the chaotropic salt is high. However, when easiness for preparing the lysis solution and precipitation of the chaotropic salt at low temperature are taken into consideration, concentration of the chaotropic salt is preferably from 0.1 to 10 mol/l, more preferably from 0.5 mol/l to 5 mol/l, most preferably from 3 mol/l to 4.5 mol/1.

When pH of the lysate solution was low, pass-through time of the lysate solution and pass-through time of the washing solution became short. Examples of the useful method for controlling pH of the lysate solution include a method in which pH is controlled with the buffer used in the dispersion medium, a method in which pH is controlled by adding a buffer to the lysis solution, a method in which a buffer is added the water-soluble organic solvent in preparing the lysate solution, a method in which a buffer is added to the washing solution, and a method in which a buffer is not added to these solutions in advance, but is prepared and added thereto later.

As the buffer agents which can be used, generally used pH buffer agents (buffers) can be exemplified. Preferably, pH buffer agents for biochemical use can be exemplified. As such a buffer agent, Bis-Tris buffer can be used.

Concentration of the buffer in the aforementioned nucleic acid-solubilizing reagent is preferably from 1 to 500 mmol/l, and regarding the pH after preparation of the lysis solution, it is desirable to use those which show preferably from pH 3 to 8, more preferably from pH 4 to 7, further preferably from pH 5 to 7.

It is desirable that the lysis solution contains a nucleic acid-stabilizing reagent. The term “nucleic acid-stabilizing agent” as used herein means a reagent which can effect stable presence of a nucleic acid in an analyte. This also includes a reagent which can effect stable presence of the nucleic acid itself and also prevent degradation of the nucleic acid by reducing or completely inhibiting in-stabilization of the nucleic acid, such as the degradation action by a nuclease or the like nucleic acid degrading enzyme that degrades the nucleic acid. It is desirable that the nucleic acid-stabilizing reagent is allowed to coexist with one or more substances selected from an organic solvent, a chaotropic salt, a surface active agent, a buffer and an antifoaming agent.

As the nucleic acid-stabilizing reagent having the action to inactivate the nuclease activity, a compound generally used as a reducing agent can be used. As the reducing agent, hydrogen, hydrogen iodide, hydrogen sulfide, lithium aluminum hydride, sodium borohydride and the like hydride compounds, an alkali metal, magnesium, calcium, aluminum, zinc and the like metals having large electropositive, or amalgam thereof, aldehydes, saccharides, formic acid, oxalic acid and the like organic oxides, a mercapto compound and the like can be exemplified. Among them, a mercapto compound is particularly desirable. As the mercapto compound, N-acetylcysteine, mercaptoethanol, alkylmercaptan and the like can be exemplified. The mercapto compounds may be used alone or as a combination of two or more.

It is desirable that concentration of the nucleic acid-stabilizing reagent in the lysis solution is from 0.01 to 20% by mass, more preferably from 0.03 to 15% by mass. Concentration of the mercapto compound in the lysis compound is preferably from 0.01 to 20% by mass, more preferably from 0.05 to 15% by mass, most preferably from 0.05 to 5% by mass. (In this specification, mass ratio is equal to weight ratio.)

In addition, the mercapto compound also has an effect to shorten pass-through time of the lysate solution and washing solution as its concentration in the lysis solution increases. However, too high concentration from the viewpoint of the worker's working environment. Also from these points, concentration of the mercapto compound in the lysis compound is preferably from 0.01 to 20% by mass, more preferably from 0.05 to 15% by mass, most preferably from 0.05 to 5% by mass.

The inventors have found that pass-through time of the lysate solution and pass-through time of the washing solution can be shortened by adding a surface active agent in the lysis solution. As the surface active agent to be added, a nonionic surface active agent, a cationic surface active agent and an amphoteric surface active agent can for example be cited.

According to the invention, a nonionic surface active agent and a cationic surface active agent can be used preferably.

As the nonionic surface active agent, a polyoxyethylene alkyl phenyl ether system surface active agent, a polyoxyethylene alkyl ether system surface active agent, a fatty acid alkanol amide can be exemplified, of which a polyoxyethylene alkyl ether system surface active agent is desirable. Among the polyoxyethylene (POE) alkyl ether system surface active agents, POE decyl ether, POE lauryl ether, POE tridecyl ether, POE alkylene decyl ether, POE sorbitan monolaurate, POE sorbitan monooleate, POE sorbitan monostearate, polyoxyethylene sorbitol tetraoleate, POE alkyl amine and POE acetylene glycol are more preferable.

As the cationic surface active agent, cetyl trimethylammonium bromide, dodecyl trimethylammonium chloride, tetradecyl trimethylammonium chloride and cetyl pyridinium chloride can be exemplified.

As a result of intensive studies on the concentration of surface active agent, pass-through time of the lysate solution and pass-through time of the washing solution were shortened as the concentration of surface active agent was increased. However, when the concentration of surface active agent was increased too much, it caused reduction of nucleic acid recovery yield depending on the kind of chaotropic salt, and a problem of generating foam was found. Based on these results, according to the invention, it is desirable to set concentration of surface active agent in the solution to a level of preferably from 0.001% by mass to 30% by mass, particularly preferably from 0.1% by mass to 7.5% by mass. In addition, since the lysate solution is apt to foam when a surface active agent is used, it may not be used or an antifoaming agent may be used, with the proviso that sufficient performance can be obtained even when the surface active agent is not used from the viewpoint of easy handling.

Examples of the antifoaming agent include a silicon system antifoaming agent (e.g., silicone oil, dimethyl polysiloxane, silicone emulsion, modified polysiloxane, silicone compound or the like), an alcohol system antifoaming agent (e.g., acetylene glycol, heptanol, ethylhexanol, higher alcohol, polyoxyalkylene glycol or the like), an ether system antifoaming agent (e.g., heptyl cellosolve, nonyl cellosolve-3-heptylsorbitol or the like), an oil and fat system antifoaming agent (e.g., an animal or plant oil or the like), a fatty acid system antifoaming agent (e.g., stearic acid, oleic acid, palmitic acid or the like), a metallic soap system antifoaming agent (e.g., aluminum stearate, calcium stearate or the like), a fatty acid ester system antifoaming agent (e.g., natural wax, tributyl phosphate or the like), a phosphorus phosphoric acid ester system antifoaming agent (e.g., sodium octyl phosphate or the like), an amine system antifoaming agent (e.g., diamylamine or the like), an amide system antifoaming agent (e.g., stearic acid amide or the like), and other antifoaming agents (e.g., ferric sulfate, bauxite and the like). These antifoaming agents may be used alone or as a combination of two or more. Particularly preferred is the use of a combination of two components of a silicon system antifoaming agent and an alcohol system antifoaming agent.

It is desirable that concentration of the antifoaming agent in the lysis solution is from 0.1 to 10% by mass.

Pass-through time of the washing solution can be sharply shortened by mixing the lysis solution with a water-soluble organic solvent. The term “water-soluble organic solvent” as used herein means a water-soluble organic solvent wherein its concentration is 100% or less under a state of dissolved in water. As a result of intensive studies, the inventors have found an water-soluble organic solvent concentration which is effective in shortening pass-through time of the lysate solution and washing solution, eluting more larger amount of nucleic acid from the extraction membrane by one elution step, and increasing recovery yield of nucleic acid.

In addition to the aforementioned effects, the solubility of various reagents contained in the lysis solution can be cited as an advantage of the addition of water-soluble organic solvent. In addition, the effect of shortening pass-through time of the lysate solution and washing solution and increasing nucleic acid recovery yield by the increase of lysis solution volume can also be cited. In the case of the extraction system of the invention which uses a cartridge, the cartridge size is fixed so that the maximum volume of the lysate solution applicable into the cartridge depends on the cartridge size. This means that it is necessary to exchange it with a more larger cartridge in order to carry out the extraction operation by exceeding the applicable maximum liquid volume calculated from the selected cartridge size, namely the lysate solution volume, thus generating a possibility of redoing designing of the extractor. With the aim of solving these problems, the inventors have conducted intensive studies and found as a result, as a lysate solution preparation method of the invention wherein the lysis solution is added to a biomaterial and then a water-soluble organic solvent is added thereto, a method in which liquid volume of this water-soluble organic solvent is reduced as many as possible, and the resulting liquid volume obtained by the reduction is used for the liquid volume increase of the lysis solution. Also from this point of view, it is important to mix the lysis solution with an water-soluble organic solvent.

As a result of conducting intensive studies on the concentration of water-soluble organic solvent in the lysis solution, it was found that pass-through time of the lysate solution and pass-through time of the washing solution are shortened and the nucleic acid recovery yield is also increased as the amount of the water-soluble organic solvent is increased. However, since the nucleic acid yield which can be eluted by one elution operation in the nucleic acid recovery step was reduced as the concentration of water-soluble organic solvent in the lysis solution was increased, it was necessary to carry out the extraction operation several times for obtaining desired yield of the nucleic acid.

Thus, since recovery efficiency of the nucleic acid is reduced when concentration of the alcohol in the lysis solution is too high, in the case of the use of an water-soluble organic solvent in the lysis solution, it is desirable that the alcohol concentration in the lysis solution is preferably 70% by volume or less, more preferably 50% by volume or less, particularly preferably 25% by volume or less.

As the kind of the water-soluble organic solvent to be added to the lysis solution, acetone, alcohols, dimethylformamide and the like can be exemplified. Among them, alcohols are desirable. The alcohols may be any one of a primary alcohol, a secondary alcohol and a tertiary alcohol. Particularly, methanol, ethanol, propanol and an isomer thereof, and butanol and an isomer thereof can be used more preferably. Among them, ethanol is particularly desirable from the viewpoint of the reduction of environmental load and toxicity. These water-soluble organic solvents may be used alone or as a combination of two or more.

In addition, the water-soluble organic solvent may not be added when pass-through time of the lysate solution and washing is sufficiently quick. As such an example, a case in which small number of cells are handled can for example be cited.

The analyte may be subjected to a homogenization treatment at a step of before addition of the lysis solution, after addition of the lysis solution or after preparation of the lysate solution. It is considered that improvement of clogging and advancement of pass-through time can be effected by carrying out the homogenization treatment, because components which delay the pass-through time, namely substances that cause clogging, are pulverized thereby. The homogenization treatment can be carried out by an ultrasonic treatment, a treatment which uses a sharp projection, a treatment which uses a high speed stirring treatment, a treatment in which the analyte is extruded from minutes voids, a treatment with a syringe equipped with a needle, a pellet pestle treatment, pipetting, a method which uses beads made of glass, stainless steel, zirconia or the like, or a combination thereof.

The homogenization method is not particularly limited. For example, in carrying out the mixing, it is desirable to treat the analyte at from 30 to 10,000 rpm for from 1 second to 3 minutes, more desirably to treat at from 300 to 7,000 rpm for from 1 second to 1 minute, most desirably to treat at from 3,000 to 6,000 rpm for from 5 seconds to 30 seconds, using a stirring apparatus.

According to the invention, it is desirable to carry out pipetting after addition of the lysis solution. For example, the pipetting is effective when cells of 1,000,000 or more are treated. When pipetting is carried out, it is desirable to carry it out simultaneously with the addition of the lysis solution using a pipette charged with the lysis solution.

Effect of the pipetting is considered as follows. Since density of the dispersed cells increases as cell density in the dispersion medium increases, a possibility of generating a moiety having high density of the structure composed of the lysed biomaterial becomes high. When this structure is formed, it is considered that the nucleic acid existing in the structure becomes difficult to be released into the lysis solution and lysate solution, concentration ratio of the chaotropic salt to the biomaterial in the structure is reduced at the same time so that lysis of the biomaterial becomes difficult to progress, and inhibitory action upon the activity of an enzyme capable of degrading the nucleic acid is also reduced so that a possibility that the released nucleic acid is degraded becomes high, so that the yield is reduced and the partially degraded cells cause clogging of the porous membrane. The reason why pipetting is carried out immediately after the addition of the lysis solution is to reduce cell density of the semi-lysed cells by pipetting or stirring before the formation of such a semi-lysed biomaterial, or even when formed, to disperse such structures by pipetting. In addition, it is considered that when an irregularity is generated in the formed state of the semi-lysed structures, it exerts influences upon the irregularity of the nucleic acid yield and irregularity of the pass-through time of the lysate solution and washing solution. The pipetting and stirring operations also have the effect to reduce these irregularities.

Based on the above, according to the invention, it is desirable to carry out pipetting when the number of cells is large, and though the frequency of pipetting is not particularly limited, it is preferable to carry out at least once or more and 50 times or less, more preferably 3 times or more and 10 times or less. In addition, when the number of cells is small, for example 5,000,000 or less, or when sufficiently quick pass-through time can be obtained with high and stable yield of the nucleic acid without carrying out the pipetting operation, pipetting may be carried out at a more smaller frequency or may not be carried out at all from the viewpoint of alleviating the user's burden and shortening the extraction operation pretreatment time.

According to the invention, it is desirable to carry out a stirring operation on a lysis solution prepared by carrying out the pipetting operation of a lysis solution prepared by lysing a biomaterial, or on the lysis solution prepared by lysing a biomaterial. As the stirring time is prolonged, pass-through time of the lysate solution and pass-through time of the washing solution are shortened, recovery yield of the nucleic acid increases and the nucleic acid recovery yield becomes stable. Regarding the period of time of stirring, when the number of cells is small, for example 1,000,000 cells or less in the case of the floating cell HL 60, these can be recovered by the step before this step, namely the pipetting step alone without stirring, or by a stirring time of 1 minute or less, so that the stirring operation may not be carried out or the stirring time may be set to 1 minute or less.

A water-soluble organic solvent is added to the lysis solution prepared by the above step, wherein nucleic acid is eluted by lysing a biomaterial, and the nucleic acid is allowed to contact with an adsorbing solid material. Said solid material is not particularly limited and nylon or the like can be used, but a solid material having hydroxyl groups on the surface is desirable. It is considered that the nucleic acid adsorption mechanism of the invention is that the nucleic acid in the sample solution is adsorbed by the surface of a solid material, particularly by an organic high polymer having hydroxyl groups on the surface, by this operation, or is captured and adsorbed by the filter surface and pores in the case of a porous membrane. According to the invention, the water-soluble organic solvent is not particularly limited, but alcohols can be suitably used. The alcohols may be any one of a primary alcohol, a secondary alcohol and a tertiary alcohol, and methanol, ethanol, propanol and an isomer thereof, and butanol and an isomer thereof can be used more preferably. These water-soluble organic solvents may be used alone or as a combination of two or more. Ethanol can be used as particularly desirable water-soluble organic solvent.

Regarding concentration of the water-soluble organic solvent to be added, the solvent is added such that its concentration at the time of preparation of the lysate solution becomes from 10% by volume to 60% by volume. When concentration of the water-soluble organic solvent is low, yield of the nucleic acid is reduced. The reason for this may be that the nucleic acid which should be kept on the membrane is transferred to the through liquid side without binding to the membrane while the lysate solution is passing through the membrane, thus causing reduction of the nucleic acid recovery yield. When concentration of the water-soluble organic solvent is high, the pass-through time becomes short, but the yield is considerably reduced. It is considered that this is because the size of substances as the cause of the clogging becomes large by aggregating under high water-soluble organic solvent concentration, and as a result, the aggregates cannot enter into pours of the membrane but accumulate on the upper side of the membrane, so that the lysate and the like solutions and washing solution become able to pass markedly easily through the spaces in the deposits formed from the precipitates.

Based on the above examination results, according to the invention, it is necessary to set concentration of the water-soluble organic solvent to a level of from 10% by volume to 60% by volume as the concentration at the time of the preparation of the lysate solution, particularly preferably from 20% by volume to 40% by volume.

After addition of the water-soluble organic solvent, it is desirable to carry out at least one pipetting operation or stirring operation or both of them. When the stirring operation is carried out, it can be carried out by once for one sample by one sample, but it is preferable from the viewpoint of the alleviation of the user's burden to carry out the stirring twice, wherein the first stirring is carried out for one analyte by one analyte, and the second stirring is carried out for all analytes. These operations are particularly effective when the number of analytes is large.

Illustratively, for example, in the case of the extraction operation on one or more analytes, stirring is carried out just after the addition of the water-soluble organic solvent to one analyte by one analyte in the first stirring, and the analytes are stirred in one lot in the second stirring. When the first stirring is carried out, it is desirable to carry out the treatment in such a manner that contacting area of the water-soluble organic solvent with the lysis solution in which a biomaterial is lysed becomes as minimum as possible, and their contacting period of time becomes minimum. Such a tendency becomes significant as the number of cells becomes large. In order to prevent such a phenomenon, it is desirable to carry out the stirring just after the addition of the water-soluble organic solvent.

Regarding the period of time of the first stirring, when the stirring time is prolonged, pass-through rate of the lysate solution and washing solution becomes quick, yield of the nucleic acid increases, and the pass-through time of the lysate solution and washing solution and the nucleic acid recovery yield are also stabilized. It is considered that this is due to dwindling of the un-lysed portions at the time when a biomaterial is lysed by adding the lysis solution or the clogging-generating substances formed by adding the water-soluble organic solvent, effected by the stirring operation.

Also in the case of the period of time of the second stirring, when the stirring time is prolonged, pass-through rate of the lysate solution and washing solution becomes quick, yield of the nucleic acid increases, and the pass-through time of the lysate solution and washing solution and the nucleic acid recovery yield are also stabilized. The reason for this is considered to be the same as the case of the first stirring.

In addition, amount of the nucleic acid eluted by one elution operation is increased as the stirring time is prolonged. For example, when the number of cells is 10,000,000 in the case of the use of HL 60 as the cell species, desired yield cannot be obtained by one extraction operation unless the stirring time is set to 1 minute or more, and when set to 1 minute or less, several times of the elution or more larger volume of extraction liquid becomes necessary.

The stirring period of time may be 0.1 second or more and 600 seconds or less for both of the first and second stirrings, and a range of 10 seconds or more and 120 seconds or less is particularly desirable. In addition, from the viewpoint of alleviating the use's burden, it is desirable to set the first stirring time to a shorter period, and the second stirring time to a longer period than that of the first stirring.

Similar to the case of the stirring operation, pass-through time of the lysate solution and washing solution becomes short and yield of the nucleic acid is improved as the number of times of pipetting is increased.

From the viewpoint of alleviating the user's burden, one stirring operation or pipetting operation may be enough when the number of the cells to be used is small.

When the number of cells is small, the cells can be sufficiently lysed and the extraction can also be effected with a further smaller volume of the lysate solution. When amount of the lysate solution is small, pass-through time of the lysate solution becomes short. In addition, recovery yield of the nucleic acid also increases. It is considered that this is caused by the increase of the nucleic acid concentration because of the increased concentration of the biomaterial in the lysate solution, and by the enlargement of the size of nucleic acid aggregates in the lysate solution thus resulting in their aptness to be captured by the porous membrane.

When the number of cells is large, more larger volume of the lysis solution is necessary for sufficiently lysing the cells. It is considered that when volume of the lysis solution, namely amount of the chaotropic salt, is insufficient, not only the cells are insufficiently lysed but also inhibitory action of the nucleic acid degrading enzyme is reduced so that degradation of the nucleic acid eluted from the biomaterial becomes highly possible and its yield is reduced.

Based on the above, according to the invention, it is desirable to use 800 μl or less of the lysate solution when the number of cells is 500,000 or less, and to use 300 μl or more of the lysate solution when the number of cells is 500,000 or more.

It is desirable that the lysate solution has a surface tension of 0.05 J/m² or less, a viscosity of from 1 to 10,000 mPa, and a specific gravity of from 0.8 to 1.2. When the solution has such ranges, the next step in which the lysate solution is allowed to pass through a solid material such as a nucleic acid adsorbing porous membrane to effect adsorption of the nucleic acid and then the residue is removed can be easily carry out, which is desirable.

According to the nucleic acid adsorbing porous membrane of the invention, a solution can pass through its inside. In this case, the term “a solution can pass through its inside” means that when a pressure difference is generated between a space contacting with one side of the membrane and a space contacting with the other side of the membrane, the solution can pass through the membrane from the high pressure space side to the low pressure space side. Alternatively, it means that when a centrifugal force is applied to the membrane, the solution can pass through the membrane toward the direction of centrifugal force.

In addition, it is desirable that the nucleic acid adsorbing porous membrane of the invention a membrane which has a hydrophilic group on the surface. The hydrophilic group means a polar group (atomic group) which can perform interaction with water, and all of the groups (atomic groups) which are concerned in the adsorption of nucleic acid are applicable thereto. As the hydrophilic group, a group having a middle degree strength of interaction with water (cf. “A group having not so strong hydrophilic property” in the item “Hydrophilic group” in ENCYCLOPAEDIA CHIMICA published by Kyoritsu Shuppan) are suitable, and its examples include hydroxyl group, carboxyl group, cyano group, oxyethylene group and the like. Preferred among them is hydroxyl group.

The term “porous membrane having a hydrophilic group” as used herein means a porous membrane in which the material itself that forms the porous membrane has a hydrophilic group, or a porous membrane into which a hydrophilic group was introduced by treating or coating the porous membrane-forming material. The material which forms a porous membrane may be either an organic substance or an inorganic substance. For example, a porous membrane in which the material itself that forms the porous membrane an organic material having a hydrophilic group, a porous membrane into which a hydrophilic group was introduced by treating a porous membrane of an organic material having no hydrophilic group, a porous membrane into which a hydrophilic group was introduced by coating a porous membrane of an organic material having no hydrophilic group with a material having a hydrophilic group, a porous membrane in which the porous membrane-forming material itself is an inorganic material having a hydrophilic group, a porous membrane into which a hydrophilic group was introduced by treating a porous membrane of an inorganic material having no hydrophilic group, a porous membrane into which a hydrophilic group was introduced by coating a porous membrane of an inorganic material having no hydrophilic group with a material having a hydrophilic group, and the like can be used. From the viewpoint of easy processing, it is desirable to use an organic high polymer or the like organic material as the material for forming a porous membrane.

As the porous membrane of a material having a hydrophilic group, porous membranes formed from polyhydroxyethyl acrylate, polyhydroxyethyl methacrylate, polyvinyl alcohol, polyvinyl pyrrolidone, polyacrylic acid, polymethacrylic acid, polyoxyethylene, acetylcellulose, a mixture of acetylcelluloses different from each other in acetyl value and the like can be exemplified, of which a porous membrane of an organic material having hydroxyl group, particularly a porous membrane consisting of an organic high polymer having hydroxyl group can be desirably used.

As the porous membrane of an organic material having hydroxyl group, a material having a polysaccharide structure is preferable, and a porous membrane of an organic high polymer consisting of a mixture of acetylcelluloses different from each other in acetyl value can be more preferably used. As the mixture of acetylcelluloses different from each other in acetyl value, a mixture of triacetyl cellulose with diacetyl cellulose, a mixture of triacetyl cellulose with monoacetyl cellulose, a mixture of triacetyl cellulose with diacetyl cellulose and monoacetyl cellulose, and a mixture of diacetyl cellulose with monoacetyl cellulose can be preferably used. A mixture of triacetyl cellulose with diacetyl cellulose can be used particularly preferably. Mixing ratio (mass ratio) of triacetyl cellulose with diacetyl cellulose is preferably from 99:1 to 1:99, more preferably from 90:10 to 50:50.

As further desirable organic materials having hydroxyl group, the saponified products of acetylcellulose described in JP-A-2003-128691 can be exemplified. The saponified product of acetylcellulose is a product obtained by saponification of a mixture of acetylcelluloses different from each other in acetyl value, and a saponified product of a mixture of triacetyl cellulose with diacetyl cellulose, a saponified product of a mixture of triacetyl cellulose with monoacetyl cellulose, a saponified product of a mixture of triacetyl cellulose with diacetyl cellulose and monoacetyl cellulose, and a saponified product of a mixture of diacetyl cellulose with monoacetyl cellulose can also be preferably used. A saponified product of a mixture of triacetyl cellulose with diacetyl cellulose can be used more preferably. Mixing ratio (mass ratio) of a mixture of triacetyl cellulose with diacetyl cellulose is preferably from 99:1 to 1:99. More preferably, mixing ratio of the mixture of triacetyl cellulose with diacetyl cellulose is from 90:10 to 50:50. In this case, the amount (density) of hydroxyl group on the porous membrane surface can be controlled by the degree of saponification treatment (saponification ratio).

In order to improve separation efficiency of nucleic acids, it is desirable that the amount (density) of hydroxyl group is large. It is desirable that saponification rate (surface saponification ratio) of the organic material obtained by a saponification treatment is 5% or more and 100% or less, more preferably 10% or more and 100% or less.

In addition, in order to increase surface area of the organic material having hydroxyl group, it is desirable to carry out a saponification treatment of the porous membrane of acetylcellulose.

The porous membrane may be a porous membrane having a front surface and a back surface symmetrical with each other, but a porous membrane having a front surface and a back surface asymmetrical with each other can be used preferably.

The term “saponification treatment” as used herein means that acetylcellulose is allowed to contact with a saponification treating liquid (e.g., sodium hydroxide aqueous solution). By this, ester group of the ester derivative of cellulose contacted with the saponification treating liquid is hydrolyzed and hydroxyl group is introduced to form regenerated cellulose. The thus prepared regenerated cellulose is different from the original cellulose in terms of the crystalline state and the like. In addition, when the saponification ratio is changed, the saponification treatment may be carried out by changing concentration of sodium hydroxide and treating time. The saponification ratio can be easily measured by XPS (e.g., it can be determined by the decreasing degree of the peak of carbonyl group).

As a method for introducing a hydrophilic group into a porous membrane of an organic material having no hydrophilic group, a graft polymer chain having the hydrophilic group in the polymer chain or its side chain can be linked to the porous membrane. There are two methods as the method for linking the graft polymer chain to the porous membrane of organic material, namely a method in which the porous membrane and graft polymer chain are allowed to undergo a chemical bonding and a method in which a compound having a polymerizable double bond is polymerized using the porous membrane as a starting point to form a graft polymer chain.

Firstly, in the method in which the porous membrane and graft polymer chain are allowed to undergo a chemical bonding, a polymer having on its terminus or side chain a functional group capable of reacting with the porous membrane is used, and they can be grafted by allowing this functional group and the functional group of the porous membrane to undergo a chemical reaction. Though the functional group capable of reacting with the porous membrane is not particularly limited with the proviso that it can react with the functional group of the porous membrane, its examples include alkoxysilane or the like silane coupling group, isocyanate group, amino group, hydroxyl group, carboxyl group, sulfonate group, phosphate group, epoxy group, allyl group, methacryloyl group, acryloyl group and the like. As a compound particularly useful as a polymer having a reactive functional group on the terminus or side chain of the polymer, a polymer having trialkoxysilyl group on the polymer terminus, a polymer having amino group on the polymer terminus, a polymer having carboxyl group on the polymer terminus, a polymer having epoxy group on the polymer terminus and a polymer having isocyanate group on the polymer terminus can be exemplified. Though the polymer to be used in this case is not particularly limited with the proviso that it has a hydrophilic group concerned in the adsorption of nucleic acid, its illustrative examples include polyhydroxyethylacrylic acid, polyhydroxyethylmethacrylic acid and salts thereof, polyvinyl alcohol, polyvinyl pyrrolidone, polyacrylic acid, polymethacrylic acid and salts thereof, polyoxyethylene and the like.

The method in which a compound having a polymerizable double bond is polymerized using the porous membrane as a starting point to form a graft polymer chain is generally called surface graft polymerization. The surface graft polymerization is a method in which an active species is applied on the a porous membrane surface by plasma irradiation, light irradiation, heating or the like method, and a compound having a polymerizable double bond is arranged to contact with the porous membrane and bonded to the porous membrane by polymerization. It is necessary that the compound useful for forming a graft polymer chain to be linked to the porous membrane has both of two characteristics of having a polymerizable double bond and having a hydrophilic group concerned in the adsorption of nucleic acid. As such a compound, any one of the compounds of polymers, oligomers and monomers having a hydrophilic group can be used, with the proviso that each has a double bond in its molecule. Particularly useful compound is a monomer having a hydrophilic group. As illustrative examples of the particularly useful monomer having a hydrophilic group, the following monomers can be cited. For example, 2-hydroxyethyl acrylate, 2-hydroxyethyl methacrylate, glycerol monomethacrylate and the like hydroxyl group-containing monomers can be used particularly preferably. In addition, acrylic acid, methacrylic acid and the like carboxyl group-containing monomers, or alkali metal salts and amine salts thereof, can also be used preferably.

As another method for introducing a hydrophilic group into a porous membrane of an organic material having no hydrophilic group, a material having a hydrophilic group can be coated. Though the material to be used in the coating is not particularly limited with the proviso that it has a hydrophilic group concerned in the adsorption of nucleic acid, a polymer of an organic material is desirable from the viewpoint of easy operations. As the polymer, polyhydroxyethylacrylic acid, polyhydroxyethylmethacrylic acid and salts thereof, polyvinyl alcohol, polyvinyl pyrrolidone, polyacrylic acid, polymethacrylic acid and salts thereof, polyoxyethylene, acetylcellulose, a mixture of acetylcelluloses different from each other in acetyl value and the like can be exemplified, of which a polymer having a polysaccharide structure is preferable.

In addition, it is also able to coat acetylcellulose or a mixture of acetylcelluloses different from each other in acetyl value on the porous membrane of an organic material having no hydrophilic group and then to subject the coated acetylcellulose or mixture of acetylcelluloses different from each other in acetyl value to a saponification treatment. In that case, it is desirable that the saponification ratio is 5% or more and 100% or less. It is more desirable that the saponification ratio is 10% or more and 100% or less.

As the porous membrane which is an inorganic material having a hydrophilic group, a porous membrane containing a silica compound can be exemplified. As the porous membrane containing a silica compound, a glass filter can be exemplified. In addition, a porous silica thin film such as the substance described in Japanese Patent No. 3058342 ca also be exemplified. This porous silica thin film can be prepared by developing a developing liquid of a cationic amphipathic substance having a bimolecular film forming ability on a substrate, preparing a multilayer bimolecular thin film of the amphipathic substance by removing the solvent from the liquid film on the substrate, allowing the multilayer bimolecular thin film to contact with a solution containing a silica compound, and then extracting and removing the aforementioned multilayer bimolecular thin film.

As the method for introducing a hydrophilic group into a porous membrane of an inorganic material having no hydrophilic group, there are two methods, namely a method in which the porous membrane and graft polymer chain having a hydrophilic group are allowed to undergo a chemical bonding and a method in which a graft polymer chain is polymerized using a monomer having a hydrophilic group containing a double bond in the molecule, using the porous membrane as a starting point.

When the porous membrane and graft polymer chain having a hydrophilic group are allowed to undergo a chemical bonding, a functional group which reacts with the terminal functional group of the graft polymer chain is introduced into the inorganic material, the graft polymer is allowed to chemically bond thereto. Also, when a graft polymer chain is polymerized using a monomer having a hydrophilic group containing a double bond in the molecule, using the porous membrane as a starting point, the functional group which becomes the starting point in polymerizing the compound having a double bond is introduced into the inorganic material.

As the graft polymer having a hydrophilic group and the monomer having a hydrophilic group containing a double bond in the molecule, the graft polymer having a hydrophilic group and monomer having a hydrophilic group containing a double bond in the molecule, described in the aforementioned method for introducing a hydrophilic group into a porous membrane of an organic material having no hydrophilic group, can be suitably used.

As another method for introducing a hydrophilic group into a porous membrane of an inorganic material having no hydrophilic group, a material having a hydrophilic group can be coated. Though the material to be used in the coating is not particularly limited with the proviso that it has a hydrophilic group concerned in the adsorption of nucleic acid, a polymer of an organic material is desirable from the viewpoint of easy operations. As the polymer, polyhydroxyethylacrylic acid, polyhydroxyethylmethacrylic acid and salts thereof, polyvinyl alcohol, polyvinyl pyrrolidone, polyacrylic acid, polymethacrylic acid and salts thereof, polyoxyethylene, acetylcellulose, a mixture of acetylcelluloses different from each other in acetyl value and the like can be exemplified.

In addition, it is also able to coat acetylcellulose or a mixture of acetylcelluloses different from each other in acetyl value on the porous membrane of an inorganic material having no hydrophilic group and then to subject the coated acetylcellulose or mixture of acetylcelluloses different from each other in acetyl value to a saponification treatment. In that case, it is desirable that the saponification ratio is 5% or more and 100% or less. It is more desirable that the saponification ratio is 10% or more and 100% or less.

As the porous membrane of an inorganic material having no hydrophilic group, a porous membrane prepared by processing aluminum or the like metal, glass, cement, pottery or the like ceramics, or new ceramics, silicon, activated carbon or the like can be exemplified.

It is desirable that the aforementioned nucleic acid-adsorbing porous membrane can pass a solution through the inside and has a thickness of from 10 μm to 500 μm. More preferably, the thickness is from 50 μm to 250 μm. It is desirable that the thickness is as thin as possible from the viewpoint of easy washing and short pass-through time of the lysate solution.

It is desirable that the aforementioned nucleic acid-adsorbing porous membrane which can pass a solution through the inside has a minimum pore size of 0.22 μm or more. More preferably, the minimum pore size is 0.5 μm or more. In addition, it is desirable to use a porous membrane having a maximum pore size/minimum pore size ratio of 2 or more. By this, sufficient surface area for the adsorption of nucleic acid is obtained, and clogging hardly occurs. More preferably the maximum pore size/minimum pore size ratio is 5 or more.

It is desirable that the aforementioned nucleic acid-adsorbing porous membrane which can pass a solution through the inside has a void volume of from 50 to 95%. More preferably, the void volume is from 65 to 80%. In addition, it is desirable that the bubble point is from 0.1 to 10 kgf/cm². More preferably, the bubble point is from 0.2 to 4 kgf/cm².

It is desirable that the aforementioned nucleic acid-adsorbing porous membrane which can pass a solution through the inside has a pressure loss of from 0.1 to 100 kPa. By this, uniform pressure is obtained at the time of overpressure. More preferably, the pressure loss is from 0.5 to 50 kPa. In this connection, the pressure loss is a minimum pressure necessary for passing water through a membrane of 100 μm in thickness.

It is desirable that the aforementioned nucleic acid-adsorbing porous membrane which can pass a solution through the inside has a water-permeability of from 1 to 5,000 ml per minute per 1 cm² membrane when water is allowed to pass through under a pressure of 1 kg/cm² at 25° C. More preferably, the water-permeability is from 5 to 1,000 ml per minute per 1 cm² membrane when water is allowed to pass through under a pressure of 1 kg/cm² at 25° C.

It is desirable that the aforementioned nucleic acid-adsorbing porous membrane which can pass a solution through the inside is a cellulose derivative which does not dissolve within 1 hour but dissolves within 48 hour when a square piece of the porous membrane having a side of 5 mm is soaked in 5 ml of trifluoroacetic acid. Also preferred is a cellulose derivative which dissolves within 1 hour when a square piece of the porous membrane having a side of 5 mm is soaked in 5 ml of trifluoroacetic acid but does not dissolve within 24 hours when soaked in 5 ml of dichloromethane. Among them, the cellulose derivative which dissolves within 1 hour when a square piece of the porous membrane having a side of 5 mm is soaked in 5 ml of trifluoroacetic acid but does not dissolve within 24 hours when soaked in 5 ml of dichloromethane is more preferable.

When the lysate solution is allowed to pass through the nucleic acid-adsorbing porous membrane, it is desirable to allow the lysate solution to pass from one side to the other side, from the viewpoint that the solution can be uniformly contacted with the porous membrane. When the lysate solution is allowed to pass through the nucleic acid-adsorbing porous membrane, it is desirable to allow the lysate solution to pass through the nucleic acid-adsorbing porous membrane from its large pore size side to small pore size side, from the viewpoint that clogging hardly occurs.

When the lysate solution is allowed to pass through the nucleic acid-adsorbing porous membrane, it is desirable that its flow rate is from 2 to 1,500 μl/sec per cm² area of the membrane in order to obtain appropriate contacting time of the solution with the porous membrane. Sufficient separation purification effect cannot be obtained when contacting time of the solution with the porous membrane is too short, and too long is also not preferable from the viewpoint of workability. It is more desirable that the aforementioned flow rate is from 5 to 700 μl/sec per cm² area of the membrane.

In addition, the nucleic acid-adsorbing porous membrane through which the solution to be used can be passed may be one, but two or more membranes can also be used. The two or more of nucleic acid-adsorbing porous membranes may be the same or different from one another.

The two or more of nucleic acid-adsorbing porous membranes may be a combination of a nucleic acid-adsorbing porous membrane of an inorganic material with a nucleic acid-adsorbing porous membrane of an organic material. For example, a combination of glass filter with a porous membrane of regenerated cellulose can be cited. In addition, the two or more of nucleic acid-adsorbing porous membranes may be a combination of a nucleic acid-adsorbing porous membrane of an inorganic material with a nucleic acid-adsorbing porous membrane of an organic material. For example, a combination of glass filter with a porous membrane of nylon or polysulfone can be cited.

A cartridge for separation and purification of nucleic acid which receives, in a container having at least two openings, the aforementioned nucleic acid-adsorbing porous membrane through which a solution can be passed can be preferably used. In addition, a cartridge for separation and purification of nucleic acid which receives, in a container having at least two openings, two or more of the aforementioned nucleic acid-adsorbing porous membrane through which a solution can be passed can be preferably used. In that case, the two or more nucleic acid-adsorbing porous membranes to be received by the container having at least two openings may be the same or different from one another.

It is desirable that the cartridge for separation and purification of nucleic acid does not receive, in the container having at least two openings, other members than the aforementioned nucleic acid-adsorbing porous membrane through which a solution can be passed. As the material of the aforementioned container, polypropylene, polystyrene, polycarbonate, polyvinyl chloride and the like plastics can be used. In addition, a biodegradable material can also be used desirably. Also, the aforementioned container may be either transparent or colored.

As the cartridge for separation and purification of nucleic acid, a cartridge for separation and purification of nucleic acid equipped with a unit for discriminating individual cartridges for separation and purification of nucleic acid can be used. As the unit for discriminating individual cartridges for separation and purification of nucleic acid, a bar code, a two dimensional bar code, a magnetic tape, an IC card and the like can be exemplified.

In addition, a cartridge for separation and purification of nucleic acid having such a structure that the nucleic acid-adsorbing porous membrane can be easily taken out from the container having at least two openings can also be used.

When soaking time of the membrane with the recovering solution is short in desorbing the nucleic acid from the membrane, it is necessary to carry out the extraction operation several times, but it is possible to elute more larger amount of the nucleic acid by one or more smaller frequency of the operation when the soaking time is prolonged. As a result of detailed examinations carried out by the inventors, it was able to obtain sufficient nucleic acid when the soaking time in extracting the nucleic acid is 0.1 second or more and 600 seconds or less.

Pass-through time of the lysate solution and washing solution becomes short when concentration of the surface active agent in the lysis solution is increased, but it becomes necessary to carry out the extraction operation several times in order to obtain desired yield of the nucleic acid, thus posing a problem of reducing concentration of the recovered nucleic acid, but more larger amount of the nucleic acid can be recovered by increasing soaking time of the nucleic acid-adsorbed membrane in the recovering solution.

Injection of the prepared lysate solution into two or more cartridges renders possible pass-through of an analyte which originally causes clogging or delays pass-through time of the lysate solution and washing solution when the number of used cartridges is smaller than that.

By the washing step, recovery yield and purity of the finally obtained RNA are improved, and liquid volume of the analyte containing necessary RNA can be minimized. In addition, when the washing and recovery operations are automated, it becomes possible to carry out the operations conveniently and quickly. The washing step may be carried out once when quickness is desired, but it is desirable to repeat the washing two or more times when the purity is more important.

In the washing step, the washing solution is supplied to the cartridge for separation and purification of nucleic acid which receives the nucleic acid-adsorbing porous membrane, using a tube, a pipette, an automatic injector or other feeding units having the same function thereof. The washing solution can be supplied from the opening 1 of the cartridge for separation and purification of nucleic acid (the opening where the nucleic acid mixture solution was injected) and discharged from an opening different from the opening 1 by allowing the solution to pass through the nucleic acid-adsorbing porous membrane where inside of the cartridge for separation and purification of nucleic acid is adjusted to a pressurized condition using a pressure difference-generating apparatus (e.g., a dropping pipette, an injector, a pump, a power pipette or the like) connected to said opening. In addition, the washing solution can also be supplied from the opening 1 and discharged from the same opening 1. It is possible also to supply and discharge the washing solution from an opening different from the opening 1 of the cartridge for separation and purification of nucleic acid where the nucleic acid mixture solution was injected. Among them, the method in which the washing solution is supplied from the opening 1 of the cartridge for separation and purification of nucleic acid and discharged from an opening different from the opening 1 by allowing the solution to pass through the nucleic acid-adsorbing porous membrane is more desirable because of the excellent washing efficiency.

It is desirable that temperature of the washing solution in the washing step is from 4 to 70° C. Further, it is more desirable to set temperature of the washing solution to room temperature. In the washing step, a stirring by mechanical vibration or ultrasonic wave can be applied to the cartridge for separation and purification of nucleic acid simultaneously with the washing step. Alternatively, the washing can be effected by carrying out centrifugation.

It is desirable that the washing solution in the washing step is a solution which contains at least either one of a water-soluble organic solvent and a water-soluble salt. It is necessary that the washing solution has a function to wash out impurities in the nucleic acid mixture solution adsorbed by the nucleic acid-adsorbing porous membrane together with the nucleic acid. To effect this, it is necessary that the washing solution is a composition which does not desorb the nucleic acid from the nucleic acid-adsorbing porous membrane but desorbs the impurities. For this purpose, since nucleic acids are slightly soluble in an alcohol and the like water-soluble organic solvents, a water-soluble organic solvent is suited for desorbing components other than nucleic acids while holding the nucleic acids. In addition, since the nucleic acid adsorbing effect is improved by the addition of a water-soluble salt, the action to selectively remove impurities and unnecessary components can be improved thereby.

As the water-soluble organic solvent to be contained in the washing solution, an alcohol can be used. As the alcohol, methanol, ethanol, isopropanol, n-propanol and butanol can be exemplified. Propanol may be either isopropanol or n-propanol, and butanol may be either straight chain or branched chain. Two or more species of these alcohols can be used. Among them, it is desirable to use ethanol.

Amount of the water-soluble organic solvent to be contained in the washing solution is preferably from 5 to 100% by mass, more preferably from 5 to 40% by mass. This range is desirable, because recovery yield of RNA can be increased without increasing its contamination with DNA, without desorbing the RNA of interest from the porous membrane, and therefore with high purity.

On the other hand, it is desirable that the water-soluble salt to be contained in the washing solution is a halide, particularly a chloride. In addition, it is desirable that the water-soluble salt is a monovalent or divalent cation, particularly preferably an alkali metal salt or an alkaline earth metal salt, of which a sodium salt and a potassium salt are preferable, and a sodium salt is most preferable.

When the water-soluble salt is contained in the washing solution, its concentration is preferably 10 mmol/l or more, and though its upper limit is not particularly limited with the proviso that it is within such a range that it does not spoil solubility of the impurities, it is preferably 1 mol/l or less, more preferably 0.1 mol/l. Further more preferably, the water-soluble salt is sodium chloride, and it is particularly desirable that sodium chloride is contained in an amount of 20 mmol/l or more.

It is desirable that the washing solution does not contain a chaotropic substance. By this, a possibility of causing contamination with the chaotropic substance in the recovery step can be reduced. When contamination with a chaotropic substance occurs at the time of the recovery step, it inhibits the enzyme reaction in carrying out RT-PCR or the like reaction, so that when a case in which an enzyme reaction or the like is carried out later on is taken into consideration, it is ideal that a chaotropic substance is not contained in the washing solution. In addition, since chaotropic substances have corrosiveness and are hazardous, management without using a chaotropic substance is markedly advantageous for testers in terms of safe test operation, also from this point of view.

In this connection, the chaotropic substances are the aforementioned urea, guanidine hydrochloride, guanidine isothiocyanate, guanidine thiocyanate, sodium isothiocyanate, sodium iodide, potassium iodide and the like.

Conventionally, since wettability of the washing solution for a cartridge or the like container is high in carrying out the washing step in the step for separation and purification of nucleic acid, the washing solution frequently remains in the container, so that the washing solution is entrapped in carrying out the recovery step and causes reduction of purity of the nucleic acid and reduction of the reactivity in the subsequent step. Thus, when adsorption and desorption of nucleic acid are carried out using a cartridge or the like container, it is important that a solution to be used in the adsorption and washing, particularly the washing solution, does not remain in the cartridge so that the washing solution does not exert influence upon the next step and thereafter.

Accordingly, in order to prevent entrapment of the washing solution of the washing step in the recovering solution of the recovery step and thereby to stop remaining of the washing solution in the cartridge to the minimum, it is desirable to set surface tension of the washing solution to less than 0.035 J/m². When the surface tension is low, wettability of the washing solution with the cartridge is improved so that the residual liquid volume can be controlled.

However, though the ratio of water can be increased for the purpose of improving the washing efficiency, surface tension of the washing solution increases in that case and the residual liquid volume increases. When surface tension of the washing solution is 0.035 J/m² or more, the residual liquid volume can be controlled by increasing water repellency of the cartridge. Droplets are formed when water repellency of the cartridge is increased, and the residual liquid volume can be controlled by dropping of the droplets. As the method for increasing water repellency, there is a means in which a silicon or the like water repellent is coated on the cartridge surface or a silicon or the like water repellent is kneaded at the time of the cartridge molding, though not particularly limited thereto.

The washing step can be simplified making use of the nucleic acid-adsorbing porous membrane of the invention. (1) Frequency of the washing solution to pass through the nucleic acid-adsorbing porous membrane may be set to once. (2) The washing step can be carried out at room temperature. (3) The subsequent step can be carried out immediately after the washing step. (4) It is possible also to combine one or two or more of the aforementioned (1), (2) and (3). In the conventional methods, a drying step was required in many cases in order to quickly remove organic solvent contained in the washing solution, but the drying step can be omitted because the nucleic acid-adsorbing porous membrane to be used in the invention is a thin film.

In the conventional method for separating and purifying RNA, there is a problem in that contamination of samples occurs in carrying out the washing step, because the washing solution frequently scatters and adheres to others. This type of contamination in the washing step can be prevented by designing shapes of the cartridge for separation and purification of nucleic prepared by sealing the nucleic acid-adsorbing porous membrane in a container having two openings and of the waste liquor container.

In order to selectively separate and purify RNA alone from a lysate solution containing DNA and RNA, this can be carried out by allowing the solution to pass through the nucleic acid-adsorbing porous membrane-received cartridge for separation and purification of nucleic acid and thereby effecting adsorption of the nucleic acid by the nucleic acid-adsorbing porous membrane (adsorption step), and then carrying out washing (washing step 1) and carrying out a step in which a DNase is allowed to act.

The DNase is not particularly limited, and any DNase can be used.

The period of time in the step in which a DNase is allowed to perform its action on the nucleic acid-adsorbing porous membrane of the cartridge for separation and purification of nucleic acid varies depending on the amount of DNA in the nucleic acid mixture solution containing DNA and RNA and the concentration of the DNase to be acted, but is preferably from 5 seconds to 360 minutes, more preferably from 30 seconds to 130 minutes. In addition, the temperature in the step in which a DNase is allowed to perform its action on the nucleic acid-adsorbing porous membrane of the cartridge for separation and purification of nucleic acid may be 4° C. or more, preferably from 10 to 50° C., and when increased reaction efficiency is desired, the reaction can also be carried out a more higher temperature such as from 50 to 70° C. In this connection, the term “a DNase is allowed to perform its action on the nucleic acid-adsorbing porous membrane” means that the part where the nucleic acid is adsorbed by the nucleic acid-adsorbing porous membrane and the DNase are allowed to undergo the reaction, and the term “on the nucleic acid-adsorbing porous membrane” includes not only on the nucleic acid-adsorbing porous membrane but also in the pores of the porous membrane, backside outlets of the pores of the membrane and the like.

In addition, the addition of DNase in the invention also has a purpose of shortening pass-through time of the washing solution or improving clogging.

Other than the DNase, any one of a protein degrading enzyme, a lipid degrading enzyme, a sugar degrading enzyme, a nucleic acid degrading enzyme and chloroform, methanol or the like organic solvent or a mixture thereof can be added. By the addition of these substances, composing components of the clogging-causing substances remained on the membrane can be degraded so that passing ability of the washing solution can be improved, pass-through time of the washing solution can be shortened and the clogging can be improved.

Their addition may be carried out after pass-through of the lysate solution, but it is more preferable to carry out washing by the washing solution several times. This is because particularly when a protein degrading enzyme, a lipid degrading enzyme, a sugar degrading enzyme or a nucleic acid degrading enzyme is used, these degrading enzymes undergo denaturation and their activities are inhibited by the influence of the chaotropic salt remained on the membrane, so that it is highly possible that the ability to degrade the clogging-causing substances is reduced and pass-through time of the washing solution does not become short.

The recovering solution is supplied to the cartridge for separation and purification of nucleic acid which receives the nucleic acid-adsorbing porous membrane, using a tube, a pipette, an automatic injector or other feeding units having the same function thereof. The recovering solution can be supplied from the opening 1 of the cartridge for separation and purification of nucleic acid (the opening where the nucleic acid mixture solution was injected) and discharged from an opening different from the opening 1 by allowing the solution to pass through the nucleic acid-adsorbing porous membrane where inside of the cartridge for separation and purification of nucleic acid is adjusted to a pressurized condition using a pressure difference-generating apparatus (e.g., a dropping pipette, an injector, a pump, a power pipette or the like) connected to said opening. In addition, the recovering solution can also be supplied from the opening 1 and discharged from the same opening 1. It is possible also to supply and discharge the recovering solution from an opening different from the opening 1 of the cartridge for separation and purification of nucleic acid where the nucleic acid mixture solution was injected. Among them, the method in which the recovering solution is supplied from the opening 1 of the cartridge for separation and purification of nucleic acid and discharged from an opening different from the opening 1 by allowing the solution to pass through the nucleic acid-adsorbing porous membrane is more desirable because of the excellent recovering efficiency.

Desorption of RNA can be carried out by adjusting volume of the recovering solution based on the volume of the nucleic acid mixture solution prepared from an analyte. Volume of the recovering solution containing the separated and purified RNA depends on the amount of the used analyte. Generally and frequently used volume of the recovering solution is from several 10 to several 100 but when amount of the analyte is extremely small or when it is desirable to separate and purify a large amount of RNA on the contrary, volume of the recovering solution can be changed within the range of from 1 μl to several 10 ml.

As the recovering solution, purified distilled water, Tris/EDTA buffer or the like can be preferably used. In addition, when the RNA recovered after the step is subjected to RT-PCR (reverse transcriptase polymerase chain reaction), a buffer solution used in the RT-PCR (e.g., an aqueous solution having respective final concentrations of KCl 75 mmol/l, Tris-HCl 50 mmol/l, MgCl2 3.0 mmol/l and DTT 10 mmol/l) can also be used.

It is desirable that pH of the recovering solution is from 1 to 10, more preferably from 2 to 7. In addition, particularly ionic strength and salt concentration exert effect on the elution of the adsorbed RNA. It is desirable that the recovering solution has an ionic strength of 500 mmol/l or less. The salt concentration is preferably 0.5 mol/l or less, more preferably 0.01 mmol/l or more and 50 mmol/l or less. In this manner, recovery yield of RNA is improved so that more larger amount of RNA can be recovered.

A recovering solution containing concentrated nucleic acid can be obtained by reducing volume of the recovering solution. It can be preferably set to (volume of recovering solution):(volume of nucleic acid mixture solution)=1:100 to 99:100, more preferably to (volume of recovering solution):(volume of nucleic acid mixture solution)=1:10 to 9:10. In this manner, nucleic acid can be easily concentrated without carrying out an operation for concentration in the post-step of the separation and purification of nucleic acid. By these methods, a method for obtaining a nucleic acid solution in which the nucleic acid is concentrated than the analyte can be provided.

As another embodiment, a recovering solution containing a desired concentration of a nucleic acid can be obtained by carrying out desorption of the nucleic acid by adjusting volume of the recovering solution, and a recovering solution containing the nucleic acid having a concentration suited for a subsequent step, for example, when RT-PCR is carried out, can be obtained. It can be preferably set to (volume of recovering solution):(volume of nucleic acid mixture solution)=1:1 to 50:1, more preferably to (volume of recovering solution):(volume of nucleic acid mixture solution)=1:5 to 5:1. In this manner, a merit of being able to avoid the troublesome concentration adjustment after separation and purification of nucleic acid can be obtained. In addition, recovery yield of nucleic acid from the porous membrane can be increased by using sufficient volume of the recovering solution. When the adsorbed nucleic acid is desorbed from a solid material by a recovering solution, it is possible to increase nucleic acid recovery yield by lengthening the time in which the solid material is soaked in the recovering solution. The soaking time of the recovering solution is preferably from 0.1 second to 1,600 seconds, and more preferably from 5 seconds to 600 seconds.

In addition, the nucleic acid can be conveniently recovered by changing temperature of the recovering solution in response to the purpose. For example, when desorption of a nucleic acid from the porous membrane is carrying out by adjusting temperature of the recovering solution to 0 to 10° C., degradation of the nucleic acid can be prevented by inhibiting the action of nucleic acid degrading enzymes without adding certain reagents which prevent its degradation by the enzymes and employing a special operation, so that a nucleic acid solution can be obtained conveniently and efficiently.

Also, when temperature of the recovering solution is adjusted to 10 to 35° C., recovery of a nucleic acid can be carried out at general room temperature so that the nucleic acid can be desorbed and separated and purified without requiring a complex step.

As still another embodiment, when temperature of the recovering solution is set to a high temperature such as from 35 to 70° C., desorption of a nucleic acid from the porous membrane can be carried out conveniently with a high recovery yield without employing a complex operation.

Injection frequency of the recovering solution is not limited and it may be once or two or more times. In general, it is carried out by one recovery when the nucleic acid is quickly and conveniently separated and purified, but when a large amount of nucleic acid is recovered, the recovering solution may be injected two or more times.

In the recovery step, it is possible to make the recovering solution of nucleic acid into such a composition that it can be used in the subsequent step. The separated and purified nucleic acid is subjected to RT-PCR (reverse transcriptase polymerase chain reaction) in many cases. In that case, it is necessary to dilute the separated and purified nucleic acid with a buffer solution suited for the RT-PCR method. When a buffer solution suited for the RT-PCR method is used in the recovering solution of the recovery step of the instant method, transition to the subsequent RT-PCR step can be made conveniently and quickly.

In addition, it is also possible to add a stabilizing agent for the purpose of preventing degradation of the nucleic acid recovered in the nucleic acid recovering solution in the recovery step. As the stabilizing agent, an antibacterial agent, an antifungal agent, a nucleic acid degradation inhibitor or the like can be added. As the nucleic acid degradation inhibitor, a nuclease inhibitor can be exemplified, EDTA and the like can be illustratively cited. In addition, as other embodiment, a stabilizing agent can be added to a recovering container in advance.

Though the recovering container to be used in the recovery step is not particularly limited, a recovering container prepared using a material having no absorption at 260 nm can be used. In this case, concentration of the recovered nucleic acid solution can be measured without transferring it to other container. As the material having no absorption at 260 nm, quartz glass and the like can for example be cited, though not limited thereto.

The aforementioned method for separating and purifying a nucleic acid from an analyte containing the nucleic acid, using a cartridge for separation and purification of nucleic acid which receives a nucleic acid-adsorbing porous membrane in a container having at least two openings and a pressure difference-generating apparatus, can be carried out using an automatic apparatus that carries out the included steps. In addition, this can be carried out using an automatic apparatus which automatically carries out the aforementioned use of a kit. By such an automatic apparatus, not only the operation can be carried out conveniently and quickly, but a certain level of nucleic acid can also be obtained independent of the worker's skill.

The following shows an example of the automatic apparatus which automatically carries out the step for separating and purifying a nucleic acid from an analyte containing the nucleic acid, using a cartridge for separation and purification of nucleic acid which receives a nucleic acid-adsorbing porous membrane in a container having at least two openings and a pressure difference-generating apparatus, but the automatic apparatus of the invention is not limited thereto.

The automatic apparatus is an apparatus which automatically carries out the separation and purification operations for selectively separating and purifying RNA, in which a solution-passable nucleic acid-adsorbing porous membrane-received cartridge for separation and purification of nucleic acid is used, a nucleic acid mixture solution containing nucleic acid is injected into said cartridge for separation and purification of nucleic acid, the nucleic acid in said nucleic acid mixture solution is allowed to be adsorbed by the aforementioned nucleic acid-adsorbing porous membrane by pressurization, a washing solution is injected into the aforementioned cartridge for separation and purification of nucleic acid to remove impurities by pressurization, a DNase is injected into the aforementioned cartridge for separation and purification of nucleic acid to effect action of the DNase on the nucleic acid-adsorbing porous membrane, the DNase is allowed to pass through inside of the nucleic acid-adsorbing porous membrane by pressurization, a washing solution is injected into the aforementioned cartridge for separation and purification of nucleic acid to remove the degraded DNA by pressurization, and then a recovering solution is injected into the aforementioned cartridge for separation and purification of nucleic acid to desorb the RNA adsorbed by the nucleic acid-adsorbing porous membrane and recover the same together with the recovering solution. It is desirable that this apparatus is equipped with a loading mechanism which holds the aforementioned cartridge for separation and purification of nucleic acid, a waste liquor container which receives discharged liquids of residues of the aforementioned nucleic acid mixture solution, DNase and washing solution and a recovering container which receives the aforementioned recovering solution that contains RNA, a compressed air-supplying mechanism for introducing compressed air into the aforementioned cartridge for separation and purification of nucleic acid and an injection mechanism for separately injecting washing solution, DNase and recovering solution into the aforementioned cartridge for separation and purification of nucleic acid.

It is desirable that the aforementioned loading mechanism is equipped with a stand which is loaded on the apparatus body, a cartridge holder which is supported by said stand in a vertically movable manner and holds the aforementioned cartridge for separation and purification of nucleic acid, and a container holder which holds the aforementioned waste liquor container and recovering container in such a manner that their position to the aforementioned cartridge for separation and purification of nucleic acid can be exchanged at the underside of said cartridge holder.

Also, it is desirable that the aforementioned compressed air-supplying mechanism is equipped with an air nozzle which ejects compressed air from the bottom part, a pressurization head which holds said air nozzle and vertically shifts the aforementioned air nozzle against the aforementioned cartridge for separation and purification of nucleic acid held in the aforementioned cartridge holder, and a locating unit which is installed in said pressurization head and locates the cartridge for separation and purification of nucleic acid on the rack of the aforementioned loading mechanism.

In addition, it is desirable that the aforementioned injection mechanism is equipped with a washing solution injection nozzle which injects the aforementioned washing solution, a DNase injection nozzle which injects the aforementioned DNase, a recovering solution injection nozzle which injects the aforementioned recovering solution, a movable nozzle carriage which holds the aforementioned washing solution injection nozzle, the aforementioned DNase injection nozzle and the aforementioned recovering solution injection nozzle and is successively movable on the cartridge for separation and purification of nucleic acid held by the aforementioned loading mechanism, a washing solution supplying pump which sucks the washing solution from a washing solution bottle containing the washing solution and supplies it into the aforementioned washing solution injection nozzle, a DNase supplying pump which sucks the DNase from a DNase bottle containing the DNase and supplies it into the aforementioned DNase injection nozzle and a recovering solution supplying pump which sucks the recovering solution from a recovering solution bottle containing the recovering solution and supplies it into the aforementioned recovering solution injection nozzle.

According to an apparatus such as the aforementioned automatic apparatus equipped with a cartridge for separation and purification of nucleic acid, a loading mechanism which holds a waste liquor container and a recovering container, a compressed air-supplying mechanism for introducing compressed air into the cartridge for separation and purification of nucleic acid and an injection mechanism for separately injecting washing solution, DNase and recovering solution into the cartridge for separation and purification of nucleic acid, a mechanism which can automatically separate and purify RNA in a nucleic acid mixture solution efficiently within a short period of time can be constructed by automatically carrying out a step for separating and purifying RNA in which a sample solution containing nucleic acid is injected into the nucleic acid-adsorbing porous membrane-received cartridge for separation and purification of nucleic acid, the nucleic acid in nucleic acid is allowed to be adsorbed by the nucleic acid-adsorbing porous membrane by pressurization, impurities are washed and discharged by injecting a washing solution, a DNase is injected into the aforementioned cartridge for separation and purification of nucleic acid to effect action of the DNase on the nucleic acid-adsorbing porous membrane, the DNase is allowed to pass through inside of the nucleic acid-adsorbing porous membrane by pressurization, a washing solution is injected into the aforementioned cartridge for separation and purification of nucleic acid to remove the degraded DNA by pressurization, and then a recovering solution is injected to desorb and recover the RNA adsorbed by the nucleic acid-adsorbing porous membrane.

In addition, when the aforementioned loading mechanism is constructed by equipping it with a stand, a vertically movable cartridge holder which holds the cartridge for separation and purification of nucleic acid, and a container holder which holds the waste liquor container and recovering container in such a manner that they can be exchanged, setting of the cartridge for separation and purification of nucleic acid and both containers and exchange of the waste liquor container and recovering container can be conveniently carried out.

Also, when the aforementioned compressed air-supplying mechanism is constructed by equipping it with an air nozzle, a pressurization head which vertically shifts said air nozzle, and a locating unit which locates the cartridge for separation and purification of nucleic acid, secure supply of compressed air can be carried out by a convenient mechanism.

In addition, when the aforementioned injection mechanism is constructed by equipping it with a nozzle carriage which can move on a washing solution injection nozzle, a DNase injection nozzle, a recovering solution injection nozzle and the cartridge for separation and purification of nucleic acid one by one, a washing solution supplying pump which sucks the washing solution from a washing solution bottle and supplies it into the washing solution injection nozzle, and a recovering solution supplying pump which sucks the recovering solution from a recovering solution bottle and supplies it into the recovering solution injection nozzle, injection of the washing solution and recovering solution can be separately carried out one by one by a convenient mechanism.

The analyte which can be used in the invention is not particularly limited, and in the field of diagnosis for example, whole blood, blood plasma, blood serum, urine, faces, semen, saliva and the like body fluids collected as analytes or a plant (or a part thereof), an animal (or a part thereof), a bacterium, a virus, a cultured cell, lysates thereof and homogenates thereof and the like biological materials become the objects. As the cultured cell, a floating cell, an adherent cell and the like can be exemplified. The floating cell is a cell which grows and propagates by floating in a culture medium without adhering to the container wall, and for example, HL 60, U 937, HeLa S3 and the like can be cited as typical cell strains. The adherent cell is a cell which grows and propagates in a culture medium by adhering to the container wall, and for example, NIH 3T3, HEK 293, HeLa, COS, CHO cells and the like can be cited as typical cell strains. As the animal (or a part thereof) which is used as an analyte, an animal tissue can be exemplified. For example, all of the tissues which constitute liver, kidney, spleen, brain, heart, lung, thymus and the like individuals, which can be collected when an animal is anatomized or by a biopsy, can be used.

It is desirable that these analytes are treated with an aqueous solution containing a reagent which lyses the cell membrane and nuclear membrane and thereby elutes nucleic acid, so-called nucleic acid-solubilizing reagent. By this, a nucleic acid mixture solution in which the cell membrane and nuclear membrane are lysed and nucleic acids are dispersed in the aqueous solution can be obtained.

According to the invention, the “nucleic acid” may be any one of single strand, double strand, triple strand and quadruple strand or any one of the mixtures thereof, and the molecular weight also have no limitation. In addition, it may be any one of DNA, RNA, modified products thereof and mixtures thereof.

### EXAMPLES

The following describes the invention further in detail based on examples, but the invention is not limited thereto.

### Inventive Example 1

### Influence of the Replacement of Dispersion Medium Upon RNA Recovery Yield and Pass-Through Time

About 10,000,000 cells of pelletized cultured cell HL 60 which had been washed with PBS, centrifuged to remove the washing solution and then cryopreserved were prepared. This pellet contains PBS. The cryopreserved pellet was thawed to carry out the treatments shown in Table 1.

TABLE 1

(a) Untreated (pellet was used as such)
(b) A 30 μl portion of 0.5 mol/l Bis-Tris (pH 6.5) was added to (a)
(c) PBS was removed as many as possible
(d) A 30 μl portion of 0.5 mol/l Bis-Tris (pH 6.5) was added to (c)

In the case of (b) and (d), the cells were dispersed by carrying out pipetting or Vortex treatment. A 610 μl of LR 001 (mfd. by Fuji Photo Film) was added thereto, and immediately thereafter, the pipetting treatment was carried out 5 times. A lysis solution consisting of 3.66 mol/l of guanidine thiocyanate, 1% by volume of 2-mercaptoethanol and 30 μl of ethanol was used. The cells were lysed, subjected to 1 minute of a stirring treatment (2,500 rpm) using CUTE MIXER CM-1000 (mfd. by EYELA) and then spun down by centrifugation.

Next, 190 μl of ethanol was added thereto and a Vortex treatment was immediately carried out for 5 seconds. In this case, the stirring treatment was carried out one sample by one sample. Thereafter, stirring (2,500 rpm) was carried out for 55 seconds using CUTE MIXER CM-1000. In this case, the stirring treatment of the 4 samples was carried out at one time. After spin-down by centrifugation, a lysate solution was prepared.

A NEXT cartridge (mfd. by Fuji Photo Film, aperture 7 mm, pore 2.5 μm), a washing solution (WRC) and a recovering solution (CRC) were set to Quick Gene 800 (mfd. by Fuji Photo Film), and then the lysate solution was put into the NEXT cartridge to carry out the extraction by the RNA mode of Quick Gene 800. In this case, volume of the washing solution was set to 500 μl, and volume of the recovering solution to 100 μl, soaking time of the recovering solution to 30 seconds, and the pressurization time, namely clogging judging time, to 120 seconds.

Determination of the recovered RNA and its purity determination were carried out by measuring it using an spectrophotometer for ultraviolet and visible region, Nanoprop (mfd. by Nanoprop Technologies), the recovery yield was determined from the absorbance at 260 nm, and purity of the nucleic acid from the ratio of 260 nm and 280 nm, and when this ratio was 1.8 or more, the purity was judged good. Contamination of DNA and the like was analyzed using a gel electrophoresis. Regarding conditions of the gel electrophoresis, TAE (Tris-acetate) was used as the buffer, 5 μl it of the sample was mixed with a loading buffer (10× Blue Juice) and then the whole volume was subjected to the electrophoresis.

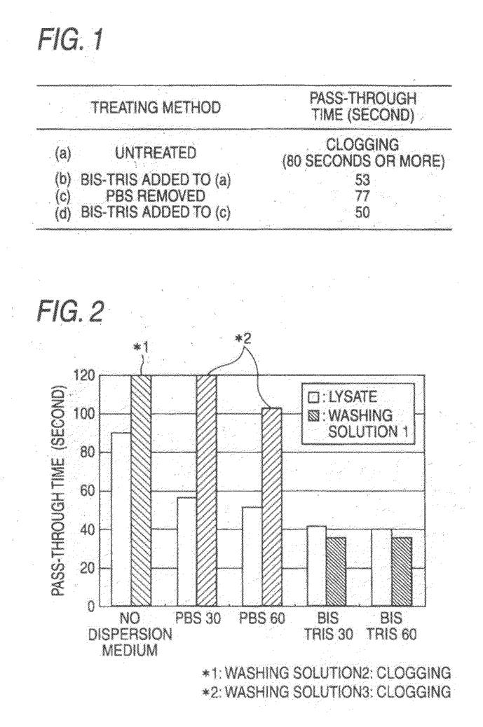

The results are shown in FIG. 1. The untreated sample of (a) generated clogging. The (b) in which PBS was removed and then Bis-Tris was added did mot cause clogging, but pass-through time of the lysate became a prolonged period of 77 seconds. Contrary to this, the (b) and (c) in which the Bis-Tris buffer solution was added, pass-through time of lysate was a level of 50 seconds which was fairly low.

### Comparative Example 1

### Relationship of the Kinds and Volume of Dispersion Medium with the RNA Recovery Yield and Pass-Through Time

About 10,000,000 cells of pelletized cultured cell HL 60, which had been cryopreserved, were thawed and mixed with a predetermined amount of PBS or 0.5 mol/l Bis-Tris (pH 6.5), and the cells were dispersed by carrying out pipetting or Vortex treatment. A 540 μl portion of a lysis solution was added thereto, and immediately thereafter, the pipetting was carried out 5 times. A lysis solution consisting of 3.66 mol/l of guanidine thiocyanate (GTC), 1% by volume of 2-mercaptoethanol and 30 μl of ethanol was used. The cells were lysed, subjected to 1 minute of a stirring treatment (2,500 rpm) using CUTE MIXER CM-1000 and then spun down by centrifugation.

Next, 230 μl of ethanol was added thereto and a Vortex treatment was immediately carried out for 5 seconds. Thereafter, stirring (2,500 rpm) was carried out for 55 seconds using CUTE MIXER CM-1000, spin-down was effected by centrifugation and then a lysate solution was prepared. However, when the number of samples is 1, the stirring time after the addition of ethanol was not divided into 5 seconds and 55 seconds, but 60 seconds of Vortex treatment was carried out immediately after the addition of ethanol to prepare the lysate solution.

RNA extraction method, calculation of recovery yield and determination of purity were carried out in the same manner in Inventive Example 1.

The results are shown in FIG. 2. When the dispersion medium was absent, pas-through time of the lysate became a prolonged period of time of 90 seconds, and clogging occurred by the second washing. When the dispersion medium was present on the other hand, pass-through time of the lysate was shortened by a factor of from about 35% to about 60%, so that the pass-through ability was sharply improved. In this connection, the condition was set such that the clogging was judged present when the pass-through time exceeded 120 seconds. Regarding the relationship between the kinds of dispersion medium and the pass-through time, the Bis-Tris buffer solution was effective in shortening pass-through time of the lysate by a factor of from about 20% to about 30%, in comparison with the case of PBS buffer solution, and pass-through time of the washing solution was sharply shortened by a factor of about 70%. Regarding the relationship between the liquid volume of dispersion medium and the pass-through time, the pass-through time was slightly shortened when volume of the dispersion medium was large, but 30 μl or more was sufficient. Based on the above, the presence of a dispersion medium is desirable, and a desirable result is obtained when Bis-Tris buffer is used as the dispersion medium and the liquid volume is at least 30 μl or more.

### Inventive Example 2

### Relationship of the Concentration of Dispersion Medium with RNA Recovery Yield and Pass-Through Time

About 10,000,000 cells of pelletized cultured cell HL 60, which had been cryopreserved, were thawed and mixed with 30 μl of 0.5 mol/l Bis-Tris (pH 6.5), and the cells were dispersed by carrying out pipetting or Vortex treatment. A 490 μl portion of a lysis solution was added thereto. A lysis solution consisting of 5 mol/l of guanidine hydrochloride (GuHCl), 1% by volume of 2-mercaptoethanol and 2.5% by volume of Tween 20 (concentration in the lysis solution) was used. The cells were lysed, subjected to 1 minute of a stirring treatment (2,500 rpm) using CUTE MIXER CM-1000 and then spun down by centrifugation.

Next, 280 μl of ethanol was added thereto and stirring (2,500 rpm) was carried out for 1 minute using CUTE MIXER CM-1000, spin-down was effected by centrifugation and then a lysate solution was prepared.

RNA extraction method, calculation of recovery yield and determination of purity were carried out in the same manner in Inventive Example 1.

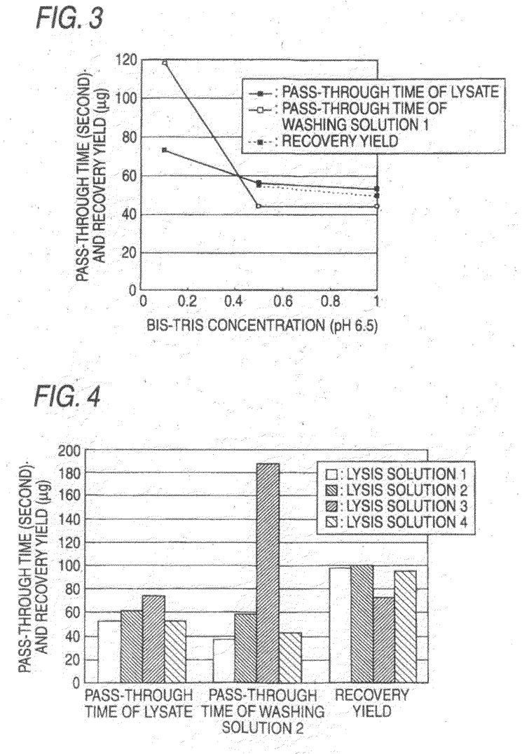

The results are shown in FIG. 3. Pass-through time of the washing solution was a considerably long time of 118 seconds when concentration of Bis-Tris was 0.1 mol/l, but was sharply shortened to about 55 seconds when concentration of Bis-Tris was 1 mol/l. In this connection, cells were not finely dispersed but aggregated.

Based on the above, it is desirable that the concentration of Bis-Tris is 0.5 mol/l.

### Inventive Example 3

### Relationship of the Kinds of Lysis Solution with RNA Recovery Yield and Pass-Through Time

About 10,000,000 cells of pelletized cultured cell HL 60, which had been cryopreserved, were thawed and mixed with 30 μl of 0.5 mol/l Bis-Tris (pH 6.5), and the cells were dispersed by carrying out pipetting or Vortex treatment. A 540 μl portion of a lysis solution was added thereto in the case of the lysis solutions 1, 3 and 4, or 510 μl of a lysis solution was added thereto in the case of the lysis solution 2, and immediately thereafter, the pipetting was carried out 5 times. Lysis solutions having the compositions shown in Table 2 were used. The cells were lysed, subjected to 1 minute of a stirring treatment (2,500 rpm) using CUTE MIXER CM-1000 and then spun down by centrifugation.

TABLE 2
<Lysis Solutions>

1. GTC (3.66 mol/l), ethanol (5.5% by volume)
2. RLT (mfd. by Qiagen)
3. GuHCl (3.66 mol/l), ethanol (5.5% by volume)
4. LR 001 (mfd. by Fuji Photo Film)

All solutions contain 1% by volume (in lysis solution) of 2-mercaptoethanol
Each concentration is concentration in lysis solution

Next, 230 μl of ethanol was added thereto in the case of the lysis solutions 1, 3 and 4, or 260 μl was added thereto in the case of the lysis solution 2, and a Vortex treatment was immediately carried out for 5 seconds. Thereafter, stirring (2,500 rpm) was carried out for 55 seconds using CUTE MIXER CM-1000, spin-down was effected by centrifugation and then a lysate solution was prepared. However, when the number of samples is 1, the stirring time after the addition of ethanol was not divided into 5 seconds and 55 seconds, but 60 seconds of Vortex treatment was carried out immediately after the addition of ethanol to prepare the lysate solution.

RNA extraction method, calculation of recovery yield and determination of purity were carried out in the same manner in Inventive Example 1.

The results are shown in FIG. 4. The lysis solution 3, namely a lysis solution constituted from GuHCl, resulted in the prolonged pass-through time of the lysate, sharply prolonged pass-through time of the washing solution, and also resulted in the reduction of recovery yield in comparison with the other cases. Particularly among the lysis solution, the lysis solution 1, namely a lysis solution constituted from GTC, showed a excellent result.

### Inventive Example 4

### Relationship of the Concentration of Chaotropic Salt in Lysis Solution with RNA Recovery Yield and Pass-Through Time

About 1,000,000 cells of pelletized cultured cell HL 60, which had been cryopreserved, were thawed and mixed with 30 μl of 0.5 mol/l Bis-Tris (pH 6.5), and the cells were dispersed by carrying out pipetting or Vortex treatment. A 350 μl portion of a lysis solution was added thereto. A lysis solution consisting of a predetermined concentration of GTC and 1% by volume of 2-mercaptoethanol (both as a concentration in the lysate) was used. The cells were lysed, subjected to 1 minute of a stirring treatment (2,500 rpm) using CUTE MIXER CM-1000 and then spun down by centrifugation.

Next, 350 μl of 70% by volume of ethanol was added thereto and stirring (2,500 rpm) was carried out for 60 seconds using CUTE MIXER CM-1000, spin-down was effected by centrifugation and then a lysate solution was prepared. RNA extraction method, calculation of recovery yield and determination of purity were carried out in the same manner in Inventive Example 1.

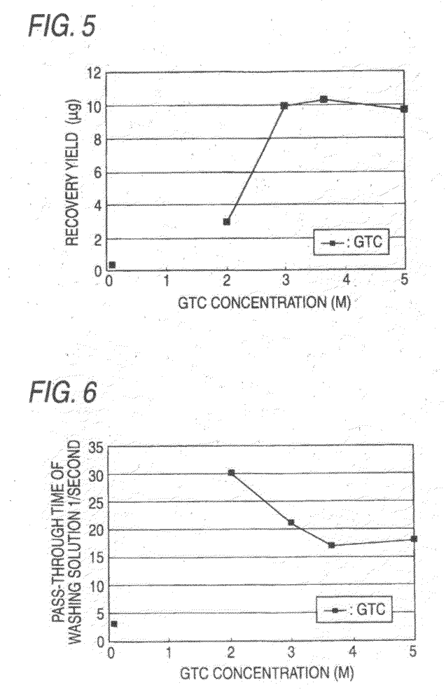

The results of recovery yield are shown in FIG. 5, and the results of pass-through time in FIG. 6. High recovery yield was obtained when the concentration of GTC was 3 mol/l or more, and the pass-through timed became short when the concentration of GTC was 3.66 mol/l or more

### Inventive Example 5

### Relationship of the volume of Lysate Solution with RNA Recovery Yield and Pass-Through Time

About 10,000,000 cells of pelletized cultured cell HL 60, which had been cryopreserved, were thawed and mixed with 30 μl of 0.5 mol/l Bis-Tris (pH 6.5), and the cells were dispersed by carrying out pipetting or Vortex treatment. A 397 μl portion (when volume of the lysate solution was 600 μl), or 469 μl (when volume of the lysate solution was 700 μl) or 540 μl (when volume of the lysate solution was 800 μl), of a lysis solution was added thereto. A lysis solution consisting of 3.66 mol/l of GTC, 1% by volume of 2-mercaptoethanol and 5.5% by volume of ethanol (each as the concentration in lysate) was used. The cells were lysed, subjected to 1 minute of a stirring treatment (2,500 rpm) using CUTE MIXER CM-1000 and then spun down by centrifugation.

Next, 173 μl (when volume of the lysate solution was 600 μl), or 202 μl (when volume of the lysate solution was 700 μl) or 230 μl (when volume of the lysate solution was 800 μl) (the concentration in lysate solution is 32.5% by volume in all cases), of ethanol was added thereto and a Vortex treatment was immediately carried out for 5 seconds. Thereafter, stirring (2,500 rpm) was carried out for 55 seconds using CUTE MIXER CM-1000, spin-down was effected by centrifugation and then a lysate solution was prepared. However, when the number of samples is 1, the stirring time after the addition of ethanol was not divided into 5 seconds and 55 seconds, but 60 seconds of Vortex treatment was carried out immediately after the addition of ethanol to prepare the lysate solution.

RNA extraction method, calculation of recovery yield and determination of purity were carried out in the same manner in Inventive Example 1.

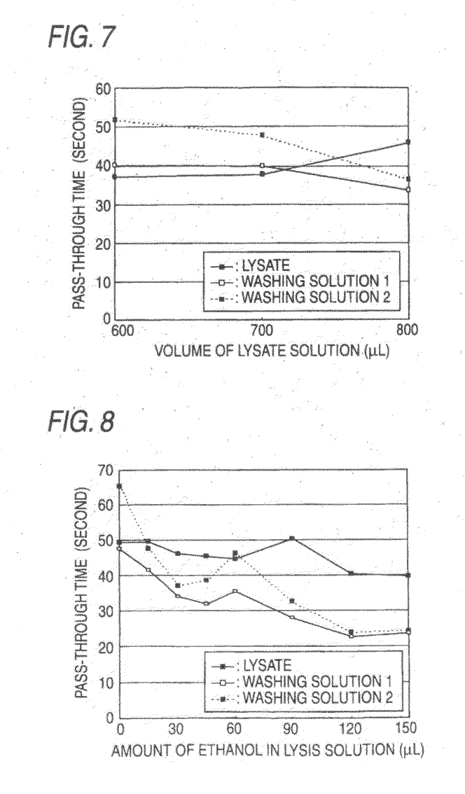

Results of the pass-through time are shown in FIG. 7. Lysate pass-through time increased as the liquid volume increased, but pas-through time of washing solution decreased. The recovery yield was almost constant regardless of the liquid volume.

### Inventive Example 6

### Relationship of the Amount of Ethanol in Lysis Solution with RNA Recovery Yield and Pass-Through Time

About 10,000,000 cells of pelletized cultured cell HL 60, which had been cryopreserved, were thawed and mixed with 30 μl of 0.5 mol/l Bis-Tris (pH 6.5), and the cells were dispersed by carrying out pipetting or Vortex treatment. From 510 μl to 770 μl of a lysis solution was added thereto. A lysis solution consisting of 3.66 mol/l of GTC, 1% by volume of 2-mercaptoethanol and from 0 to 260 μl of ethanol (concentration in lysate in all cases) was used. The cells were lysed, subjected to 1 minute of a stirring treatment (2,500 rpm) using CUTE MIXER CM-1000 and then spun down by centrifugation.

Next, from 260 μl to 0 μl of ethanol was added thereto (concentration in the lysate solution is 32.5% by volume in all cases) and a Vortex treatment was immediately carried out for 5 seconds. Thereafter, stirring (2,500 rpm) was carried out for 55 seconds using CUTE MIXER CM-1000, spin-down was effected by centrifugation and then a lysate solution was prepared. However, when the number of samples is 1, the stirring time after the addition of ethanol was not divided into 5 seconds and 55 seconds, but 60 seconds of Vortex treatment was carried out immediately after the addition of ethanol to prepare the lysate solution.

RNA extraction method, calculation of recovery yield and determination of purity were carried out in the same manner in Inventive Example 1.

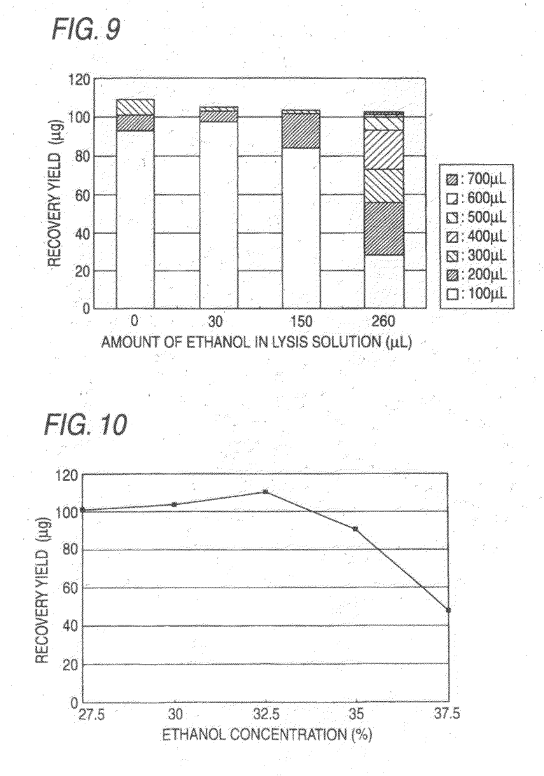

Results of the pass-through time are shown in FIG. 8. Pass-through time of the washing solution became short when the amount of ethanol in the lysis solution was 30 μl and became long with 60 μl, and then the pass-through time became short as the volume % of ethanol in the lysis solution was increased. Relationship between the ethanol concentration in the lysis solution and the recovery yield is shown in FIG. 9. An almost constant recovery yield of about 110 μg was obtained at every amount of ethanol, but with a result that more larger liquid volume of the recovering solution was required when the amount of ethanol was small or large.

Based on the above, it is desirable that amount of ethanol in the lysis solution is 30 μl.

### Inventive Example 7

### Relationship of the Amount of Ethanol in Lysate with RNA Recovery Yield and Pass-Through Time

About 10,000,000 cells of pelletized cultured cell HL 60, which had been cryopreserved, were thawed and mixed with 30 μl of 0.5 mol/l Bis-Tris (pH 6.5), and the cells were dispersed by carrying out pipetting or Vortex treatment. From 580 μl 500 μl of a lysis solution was added thereto. A lysis solution consisting of 3.66 mol/l of GTC, 1% by volume of 2-mercaptoethanol and 30 μl of ethanol (concentration in lysate in each case) was used. The cells were lysed, subjected to 1 minute of a stirring treatment (2,500 rpm) using CUTE MIXER CM-1000 and then spun down by centrifugation.

Next, from 190 μl to 270 μl of ethanol was added thereto (concentration in the lysate solution is from 27.5 to 37.5% by volume) and a Vortex treatment was immediately carried out for 5 seconds. Thereafter, stirring (2,500 rpm) was carried out for 55 seconds using CUTE MIXER CM-1000, spin-down was effected by centrifugation and then a lysate solution was prepared. However, when the number of samples is 1, the stirring time after the addition of ethanol was not divided into 5 seconds and 55 seconds, but 60 seconds of Vortex treatment was carried out immediately after the addition of ethanol to prepare the lysate solution.

RNA extraction method, calculation of recovery yield and determination of purity were carried out in the same manner in Inventive Example 1.

Results of the recovery yield of RNA are shown in FIG. 10. The recovery yield was most high when the concentration of ethanol in the lysate was 32.5% by volume.

### Inventive Example 8

### Relationship of the Stirring Time After Addition of Ethanol with Recovery Yield of RNA and Pass-Through Time

About 10,000,000 cells of pelletized cultured cell HL 60, which had been cryopreserved, were thawed and mixed with 30 μl of 0.5 mol/l Bis-Tris (pH 6.5), and the cells were dispersed by carrying out pipetting or Vortex treatment. A 540 μl portion of a lysis solution was added thereto. A lysis solution consisting of 3.66 mol/l of GTC, 1% by volume of 2-mercaptoethanol and 30 μl of ethanol (concentration in lysate in each case) was used. The cells were lysed, subjected to 1 minute of a stirring treatment (2,500 rpm) using CUTE MIXER CM-1000 and then spun down by centrifugation.

Next, 230 μl of ethanol was added thereto (concentration in the lysate solution is 32.5% by volume) and a Vortex treatment was immediately carried out for 5 seconds. Thereafter, stirring (2,500 rpm) was carried out for a predetermined period of time using CUTE MIXER CM-1000, spin-down was effected by centrifugation and then a lysate solution was prepared. However, when the number of samples is 1, the stirring time after the addition of ethanol was not divided into 5 seconds and 55 seconds, but 60 seconds of Vortex treatment was carried out immediately after the addition of ethanol to prepare the lysate solution.

RNA extraction method, calculation of recovery yield and determination of purity were carried out in the same manner in Inventive Example 1.

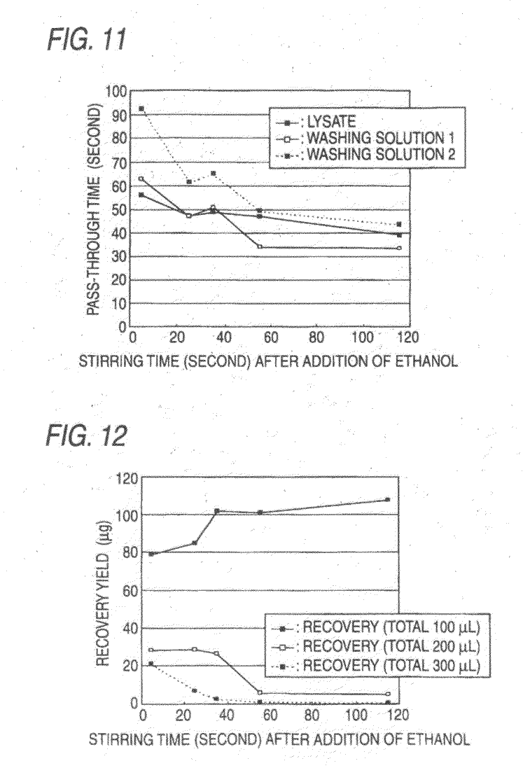

Results of the pass-through time are shown in FIG. 11. The pass-through time became short as the stirring time after the addition of ethanol was prolonged. A high level of the recovery yield of RNA was maintained when the stirring time after the addition of ethanol was 35 seconds or more, but the nucleic acid was eluted by a stirring time of 35 seconds even when the volume of recovering solution was 200 μl. (FIG. 12), so that it is desirable to carry out 55 seconds or more of the stirring after the addition of ethanol.

### Inventive Example 9

### Influence of a Difference Between Pipetting Treatment and Stirring Treatment After the Addition of Ethanol Upon the Recovery Yield of RNA and Pass-Through Time

About 10,000,000 cells of pelletized cultured cell HL 60, which had been cryopreserved, were thawed and mixed with 30 μl of 0.5 mol/l Bis-Tris (pH 6.5), and the cells were dispersed by carrying out pipetting or Vortex treatment. A 540 μl portion of a lysis solution was added thereto. A lysis solution consisting of 3.66 mol/l of GTC, 1% by volume of 2-mercaptoethanol and 30 μl of ethanol (concentration in the lysate in each case) was used. The cells were lysed, subjected to 1 minute of a stirring treatment (2,500 rpm) using CUTE MIXER CM-1000 and then spun down by centrifugation.

Next, 230 μl of ethanol was added thereto (concentration in the lysate solution is 32.5% by volume) and 5 second of Vortex treatment or 5 times of pipetting treatment was immediately carried out. Thereafter, stirring (2,500 rpm) was carried out for 55 seconds using CUTE MIXER CM-1000, spin-down was effected by centrifugation and then a lysate solution was prepared.

RNA extraction method, calculation of recovery yield and determination of purity were carried out in the same manner in Inventive Example 1.

In both of the cases in which the pipetting treatment was carried out and the stirring treatment was carried out, pass-through time of the washing solution was almost the same value of about 70 seconds. However, while the recovery yield of RNA was 75 μg in the case of carrying out the pipetting, the recovery yield of RNA in the case of carrying out the stirring was sharply increased to a value of 104 μg.

Based on the above, it is more desirable to carry out the stirring than the pipetting operation, after the addition of ethanol.

### Inventive Example 10

### Relation Ship of the Pore Size of Filter with Pass-Through Time and Recovery Yield

About 10,000,000 cells of pelletized cultured cell HL 60, which had been cryopreserved, were thawed on ice and mixed with 30 μl of 0.5 mol/l Bis-Tris (pH 6.5), and the cells were dispersed by carrying out pipetting or Vortex treatment. A 540 μl portion of a lysis solution was added thereto. A lysis solution consisting of 3.66 mol/l of GTC, 1% by volume of 2-mercaptoethanol and 30 μl of ethanol (concentration in lysate in each case) was used. The cells were lysed, subjected to 1 minute of a stirring treatment (2,500 rpm) using CUTE MIXER CM-1000 and then spun down by centrifugation.

Next, 230 μl of ethanol was added thereto (concentration in the lysate solution is 32.5% by volume) and a Vortex treatment was immediately carried out for 5 seconds. Thereafter, stirring (2,500 rpm) was carried out for 55 seconds using CUTE MIXER CM-1000, spin-down was effected by centrifugation and then a lysate solution was prepared.

RNA extraction method, calculation of recovery yield and determination of purity were carried out in the same manner in Inventive Example 1.

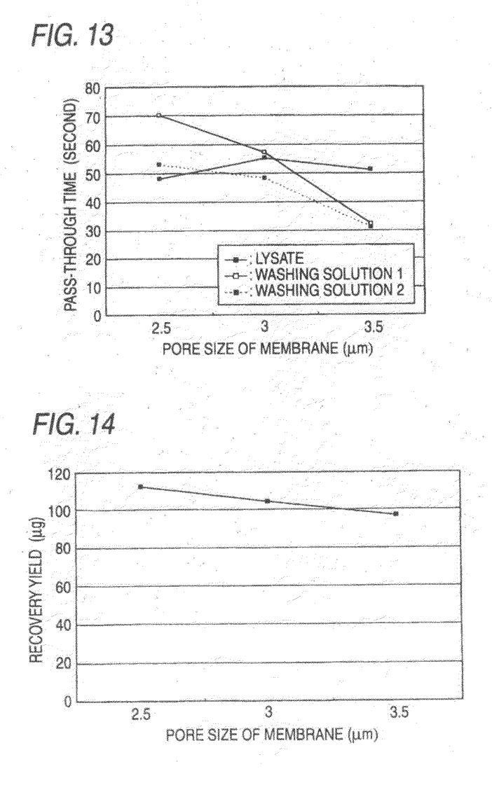

Results of the pass-through time are shown in FIG. 13. Though pass-through time of the lysate was slightly increased as the pore size was increased, pass-through time of the washing solution was sharply shortened. Results of the recovery yield are shown in FIG. 14. When the pore size was increased, recovery yield of RNA was reduced by a factor of 15%.

### Inventive Example 11

### Relationship of a Case of Using Two Cartridges with Pass-Through Time and Recovery Yield

About 10,000,000 cells of pelletized cultured cell HL 60, which had been cryopreserved, were thawed and mixed with 30 μl of 0.5 mol/l Bis-Tris (pH 6.5), and the cells were dispersed by carrying out pipetting or Vortex treatment. A 1,000 μl portion of a lysis solution was added thereto. A lysis solution consisting of 3.66 mol/l of GTC, 1% by volume of 2-mercaptoethanol and 55 μl of ethanol (concentration in lysate in each case) was used. The cells were lysed, subjected to 1 minute of a stirring treatment (2,500 rpm) using CUTE MIXER CM-1000 and then spun down by centrifugation.

Next, 400 μl of ethanol was added thereto (concentration in the lysate solution is 32.5% by volume) and a Vortex treatment was immediately carried out for 5 seconds. Thereafter, stirring (2,500 rpm) was carried out for 55 seconds using CUTE MIXER CM-1000, spin-down was effected by centrifugation and then a lysate solution was prepared.

Two NEXT cartridges, a washing solution (WRC) and a recovering solution (CRC) were set to Quick Gene 800, and then 500 μl of the lysate solution was put into each NEXT cartridge to carry out the extraction by the RNA mode of Quick Gene 800. In this case, soaking time of the recovering solution was set to 30 seconds.

Calculation of the recovery yield of RNA and determination of purity were carried out in the same manner in Inventive Example 1.

When one cartridge was used (volume of the lysate solution is 800 μl), pass-through time of the lysate was 50 seconds, but when two cartridges were used, the pass-through times were sharply reduced to 31 seconds and 34 seconds, respectively. Pass-through time of the washing solution was about 70 seconds when one cartridge was used, but when two cartridges were used, the pass-through time was sharply reduced to 20 seconds or less. The recovery yield was 104 μg in the case of 1 cartridge, but in the case of 2 cartridges, they are 50 μg and 54 μg, or a total of 104 μg, thus showing the same result of the case of 1 cartridge.

### Inventive Example 12

### Relationship of a case of using adherent cells Hek 293 and HeLa with pass-through time and recovery yield

Each of the adherent cells Hek 293 (the number of cells, about 1,700,000) and HeLa (the number of cells, about 1,500,000) was cultured on a dish of 3.5 cm, the culture medium was removed by suction, 1 ml of PBS was added thereto and softly shaken, and then the solution on the dish was removed by suction. A 540 μl portion of a lysis solution was added thereto. A lysis solution consisting of 3.66 mol/l of GTC, 1% by volume of 2-mercaptoethanol and 30 μl of ethanol (concentration in the lysate in each case) was used. The cells adhered to the dish were stripped off from the dish by rubbing with the backside of a pipette tip, and the cells were lysed. The lysis solution was transferred into a 1.7 ml capacity Eppendorf tube, subjected to 1 minute of stirring treatment (2,500 rpm) using CUTE MIXER CM-1000 and then spun down by centrifugation.

Next, 230 μl of ethanol was added thereto (concentration in the lysate solution is 32.5% by volume) and a Vortex treatment was immediately carried out for 5 seconds. Thereafter, stirring (2,500 rpm) was carried out for 55 seconds using CUTE MIXER CM-1000, spin-down was effected by centrifugation and then a lysate solution was prepared.

RNA extraction method, calculation of recovery yield and determination of purity were carried out in the same manner in Inventive Example 1.

The pass-through times of Hek 293 and HeLa were 27 seconds and 25 seconds, respectively. Pass-through times of the washing solutions were 9 seconds and 11 seconds. Recovery yields of RNA were 30 μg and 39 μg, respectively. On the other hand, by the extraction method which uses a centrifuge and a filter consisting of silicon dioxide as the main component, it was able to obtain only sharply smaller recovery yields of 20 μg and 19 μg, respectively, than those by the method of the invention.

### Inventive Example 13

### Relationship Between Recovering Solution Soaking Time and Recovery Yield

About 20,000,000 cells of pelletized cultured cell HL 60, which had been cryopreserved, were thawed and mixed with 30 μl of 0.5 mol/l Bis-Tris (pH 6.5), and the cells were dispersed by carrying out pipetting or Vortex treatment. A 630 μl portion of a lysis solution was added thereto. A lysis solution consisting of 3.66 mol/l of GTC, 1% by volume of 2-mercaptoethanol, 120 μl of ethanol and 2.5% by volume of Tween 80 (concentration in the lysate in each case) was used. The cells were lysed, subjected to 1 minute of a stirring treatment (2,500 rpm) using CUTE MIXER CM-1000 and then spun down by centrifugation.

Next, 140 μl of ethanol was added thereto (concentration in the lysate solution is 32.5% by volume) and a Vortex treatment was immediately carried out for 5 seconds. Thereafter, stirring (2,500 rpm) was carried out for 55 seconds using CUTE MIXER CM-1000, spin-down was effected by centrifugation and then a lysate solution was prepared.

A NEXT cartridge (aperture 7 mm, pore 3.5 μm), a washing solution (WRC) and a recovering solution (CRC) were set to Quick Gene 800, and then the lysate solution was put into the NEXT cartridge to carry out the extraction by the RNA mode of Quick Gene 800. In this case, soaking time of the recovering solution was set to 30 seconds or 120 seconds.

When soaking time of the recovering solution was 30 seconds, only 48 μg can be recovered with 100 μl of the recovering solution, but when soaking time of the recovering solution was 120 seconds, the recovery yield of RNA was sharply increased to 98 μg.

### Inventive Example 14

### Relationship Between the Volume of Lysate Solution and the Recovery Yield

About 10,000,000 cells of pelletized cultured cell HL 60, which had been cryopreserved, were thawed and mixed with 30 μl of 0.5 mol/l Bis-Tris (pH 6.5), and the cells were dispersed by carrying out pipetting or Vortex treatment. From 25 to 1,500 μl of a lysis solution was added thereto. A lysis solution consisting of 3.66 mol/l of GTC, 1% by volume of 2-mercaptoethanol and 30 μl of ethanol was used. The cells were lysed, subjected to 1 minute of a stirring treatment (2,500 rpm) using CUTE MIXER CM-1000 and then spun down by centrifugation.

This solution was mixed with 25 to 1,500 μl of 70% ethanol to an ethanol concentration in lysate of 35% (total liquid volume, 50 to 3,000 μl), stirring (2,500 rpm) was carried out for 5 seconds using CUTE MIXER CM-1000, and spin-down was effected by centrifugation to prepare a lysate solution.

RNA extraction method, calculation of recovery yield and determination of purity were carried out in the same manner in Inventive Example 1.

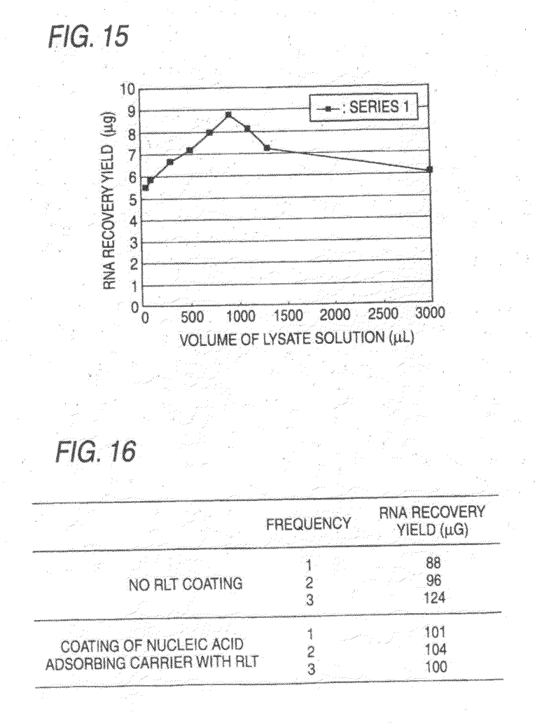

The results are shown in FIG. 15. The recovery yield of RNA increased as the volume of the lysate solution was increased, and a predicted recovery yield of 0.9 μg was obtained with a lysate liquid volume of 900 μl, but the recovery yield was reduced when the lysate liquid volume was further increased.

### Inventive Example 15

### Recovery Yield Dependency when a Solid Phase is Coated with Lysis Solution in Advance

A solid material for nucleic acid adsorption use was coated with RLT (mfd. by Qiagen) by putting 600 μl of RLT containing 1% by volume of 2-mercaptoethanol into RNeasy mini column (mfd. by Qiagen), and centrifuging it at 10,000 rpm for 15 seconds. In addition, uncoated RNeasy mini column was also prepared.

An adherent cell Hek 293 (the number of cells, about 5,800,000) was cultured on a dish of 6 cm in diameter, the culture medium was removed by suction, 1 ml of PBS was added thereto and softly shaken, and then the solution on the dish was removed by suction. A 600 μl portion of the cell lysis solution RLT containing 1% by volume of 2-mercaptoethanol was added thereto, the cells adhered to the dish were stripped off from the dish by rubbing with the backside of a pipette tip, and the cells were simultaneously lysed. This lysis solution was put into Shredder mini column (mfd. by Qiagen) and centrifuged at 15,000 rpm for 2 minutes. A 600 μl portion of 70% ethanol was added to a passing-through solution, and pipetting was carried out 7 times.

The lysate solution prepared in this manner was put into RNeasy mini column coated with RLT or uncoated RNeasy mini column and centrifuged at 10,000 rpm for 15 seconds.

The passing-through solution was discarded, and 700 μl of RW1 solution (mfd. by Qiagen) was added to the RNeasy mini column which was subsequently centrifuged at 10,000 rpm for 15 seconds. The passing-through solution was discarded, and 500 μl of RPE solution (mfd. by Qiagen) was added to the RNeasy mini column which was subsequently centrifuged at 10,000 rpm for 15 seconds. Again, the passing-through solution was discarded, and 500 μl of the RPE solution was added to the RNeasy mini column which was subsequently centrifuged at 10,000 rpm for 2 minutes.

A 50 μl portion of a DEPC (diethyl pyrocarbonate) solution was added to the RNeasy mini column and centrifuged at 10,000 rpm for 1 minute to recover the passing-through solution. A 50 μl portion of the DEPC solution was again added to the RNeasy mini column and centrifuged at 10,000 rpm for 1 minute to recover the passing-through solution.

Calculation of RNA recovery yield and determination of purity were carried out in the same manner in Inventive Example 1.

The results are shown in FIG. 16.

The solid material for nucleic acid adsorption use coated in advance with RLT showed an almost constant RNA recovery yield of 100 μg. However, though the uncoated material showed an average recovery yield of 100 μg, the value varied from 88 μg to 124 μg.

Thus, addition of the lysis solution in advance to a solid material for nucleic acid adsorption use is useful for stabilizing recovery yield of RNA.

### INDUSTRIAL APPLICABILITY

Since a nucleic acid in an analyte can be absorbed by the surface of a solid phase, the nucleic acid can be separated, purified and extracted by desorbing it via washing and the like, conveniently and quickly without requiring a special technique, a complex operation and a special device. In addition, a nucleic acid extraction method which can effect pass-through of a lysate solution and washing solution without causing clogging of cell species tat are apt to generate clogging, with the more larger number of the cells and more shorter period of time than the conventional methods, and which is high speed in comparison with the conventional methods based on the same number of cells, can be provided by the extraction method of the invention.

The entire disclosure of each and every foreign patent application from which the benefit of foreign priority has been claimed in the present application is incorporated herein by reference, as if fully set forth.

1. A method for extracting a nucleic acid, which comprises:
(a) preparing a biomaterial containing a solution by a following step (i) or (ii):
(i) a step in which a biomaterial containing a phosphate buffer solution or a Bis-Tris (N,N-bis(2-hydroxyethyl)iminotris(hydroxymethyl)methane) buffer solution is prepared; or
(ii) a step in which a buffer solution contained in a biomaterial is replaced with a Bis-Tris buffer solution;
(b) dissolving the biomaterial by allowing the biomaterial to contact with a lysis solution, and eluting a nucleic acid contained in the biomaterial;
(c) preparing a lysate solution by adding a water-soluble organic solvent to the nucleic acid-eluted solution obtained in the step (b);
(d) allowing the nucleic acid contained in the lysate solution to be adsorbed by a solid material by allowing the lysate solution to contact with the solid material;
(e) washing impurities remaining in the solid material, other than the nucleic acid to be extracted, and the lysis solution; and
(f) desorbing the absorbed nucleic acid from the solid material by a recovering solution.

2. The nucleic acid extraction method according to claim 1,
wherein the solution in the step (a) is a dispersion medium.

3. The nucleic acid extraction method according to claim 1,
wherein the solution in the step (a) has a concentration of from 0.01 to 10 mol/l and pH of from 3 to 9.

4. The nucleic acid extraction method according to any of claim 1,
wherein the lysis solution in the step (b) contains a chaotropic salt.

5. The nucleic acid extraction method according to claim 4,
wherein a concentration of the chaotropic salt is from 0.1 to 10 mol/l.

6. The nucleic acid extraction method according to any of claim 1,
wherein the lysis solution in the step (b) contains a water-soluble organic solvent in a concentration of 50% by volume or less.

7. The nucleic acid extraction method according to claim 6,
wherein the water-soluble organic solvent contained in the lysis solution is methanol, ethanol, isopropanol or butanol.

8. The nucleic acid extraction method according to claim 1,
wherein the lysis solution in the step (b) contains a surface active agent.

9. The nucleic acid extraction method according to claim 8,
wherein a concentration of the surface active agent contained in the lysis solution is from 0.001 to 30% by mass.

10. The nucleic acid extraction method according to claim 1,
wherein at least one pipetting operation is carried out after adding the lysis solution in the step (b).

11. The nucleic acid extraction method according to claim 1,
wherein stirring is carried out after the addition of the lysis solution or after the pipetting operation in the step (b).

12. The nucleic acid extraction method according to claim 1,
wherein the lysate solution in the step (c) is prepared by adding a water-soluble organic solvent to the nucleic acid-containing lysis solution so that the lysate solution contains the water-soluble organic solvent in a concentration of from 10% by volume to 60% by volume.

13. The nucleic acid extraction method according to claim 12,
wherein the water-soluble organic solvent to be used in the step (c) is methanol, ethanol, isopropanol or butanol.

14. The nucleic acid extraction method according to claim 1,
wherein at least one pipetting operation or stirring is carried out in the step (c) after the addition of the water-soluble organic solvent.

15. The nucleic acid extraction method according to claim 14,
wherein a stirring time is from 0.1 to 600 seconds.

16. The nucleic acid extraction method according to claim 14,
wherein after adding the water-soluble organic solvent and carrying out stirring or pipetting in the step (c), pipetting or stirring is further carried out.

17. The nucleic acid extraction method according to claim 16,
wherein a stirring time is from 0.1 to 600 seconds.

18. The nucleic acid extraction method according to claim 1,
wherein a soaking time of the recovering solution in the step (f) is from 0.1 second to 1,600 seconds.

19. The nucleic acid extraction method according to any claim 1,
wherein when the number of cells is 500,000 or less, a liquid amount of the lysate solution to be used is 800 μl or less.

20. The nucleic acid extraction method according to claim 1,
wherein when the number of cells is 500,000 or more, a liquid amount of the lysate solution to be used is 300 μl or more.

21. The nucleic acid extraction method according to claim 1,
wherein the solid material in the step (d) is a solid material that has a hydroxyl group on a surface of the solid material.

22. The nucleic acid extraction method according to claim 1,
wherein a container in which the solid material is kept in a cartridge is used in the step (d).

23. The nucleic acid extraction method according to claim 1,
wherein the lysate solution is allowed to contact with the solid material through which a solution containing a chaotropic salt is passed in advance in the step (d).

24. The nucleic acid extraction method according to claim 1,
wherein an extraction is carried out by injecting the lysate solution into two or more of containers in the step (c).

25. The nucleic acid extraction method according to claim 1,
wherein the lysate solution is put twice or more into one cartridge in the step (c).

26. The nucleic acid extraction method according to claim 1,
wherein in the step (d), the step (e) and the step (f), at least one of the lysate solution, the washing solution and the recovering solution is allowed to contact with the solid material by a change of pressure or centrifugation.

27. The nucleic acid extraction method according to claim 1,
wherein the nucleic acid is one of DNA, RNA, mRNA and a plasmid or a mixture thereof.

28. The nucleic acid extraction method according to claim 1,
wherein the biomaterial is a cultured cell, an animal cell, an animal tissue, a plant cell, a plant tissue, a virus, a bacterium, a fungus or a nucleic acid.
